(19) United States
(12) Patent Application Publication
(10) Pub. No.: US 2025/0069684 A1
(43) Pub. Date: Feb. 27, 2025

(54) SYSTEMS AND METHODS FOR INFERENCE OF BIOLOGICAL NETWORKS FOR BIOLOGICAL HYPOTHESIS DISCOVERY AND CLINICAL DECISION MAKING

(71) Applicant: REGENTS OF THE UNIVERSITY OF MICHIGAN, Ann Arbor, MI (US)

(72) Inventors: Visweswaran Ravikumar, Ann Arbor, MI (US); Salar Fattahi, Ann Arbor, MI (US); Arvind Rao, Ann Arbor, MI (US); Tong Xu, Ann Arbor, MI (US)

(21) Appl. No.: 18/815,472

(22) Filed: Aug. 26, 2024

Related U.S. Application Data
(60) Provisional application No. 63/534,765, filed on Aug. 25, 2023.

(51) Int. Cl. G16B 5/00; G16B 40/30; G16H 20/10

(52) U.S. Cl. CPC G16B 5/00; G16B 40/30; G16H 20/10

(57) ABSTRACT

Systems for inferring spatially-varying regulatory networks, such as gene regulatory networks, are provided. Optimization processes are performed and include regularization minimization processes that rely on one or both of a smoothly-changing minimization process and a sparsely-changing minimization process, for example to learn sparsely- or smoothly-changing Gaussian Markov random fields (GMRF). The systems are further able to convert spatially-varying data, such as spatial transcriptomic data, into spatio-temporal regulatory networks.

### CROSS-REFERENCE TO RELATED APPLICATIONS

This application claims priority to U.S. Provisional Patent Application No. 63/534,765, filed Aug. 25, 2023, the entire disclosure of which is incorporated herein by reference.

### BACKGROUND

Ribonucleic Acid (RNA) sequencing, known as RNASeq, technology has undergone rapid development in recent years, significantly impacting the amount of data generated on the transcriptome of cells. The transcriptome refers to the set of all RNA transcripts, including coding transcripts and non-coding transcripts, in an individual or a population of cells. The transcriptome is studied to examine and understand the incredibly complex set of biological systems of cell operation. Transcriptomics may be used to study the pathways that regulate varied processes of cellular function that are intricate and interconnected with varied roles in driving biological processes and disease conditions.

Recently, spatial transcriptomic techniques have been developed. Spatial transcriptomics allows clinicians to simultaneously analyze the spatial distribution of gene expression within a tissue sample. The technology combines traditional RNA sequencing with spatial information to provide a more detailed understanding of gene expression within a tissue. The technique involves capturing the spatial location of RNA molecules within tissue sections by converting the RNA molecules into spatially coded information. From this coded information, the resulting data can be used to create a spatial map of gene expression within the tissue. Spatial transcriptomics based on RNASeq data allows investigators to capture spatial transcriptomic data across a tissue sample and then analyze that data across pathways to identify and predict gene interactions and relationships. Using RNASeq data to examine gene interactions on different pathways one can, theoretically, put together an overarching network of the interactions involved in intracellular signaling and regulation would allow for more predictive modeling of cellular states (i.e., homeostasis, aging, disease). Spatial transcriptomics has numerous applications in fields such as developmental biology, cancer research, and neuroscience. It can be used to study how genes are regulated during tissue development, how different cell types interact within a tissue, and how gene expression changes in response to disease or environmental factors.

The advent of high throughput sequencing technologies has transformed our understanding of biological systems and catalyzed the adoption of a systems-level approach to studying biological processes. Networks have emerged as the intuitive framework for reasoning about complex biological systems. Nodes in the network represent individual components, and edges represent direct interactions between them. A particular use of transcriptomic data is the ability to generate gene regulatory networks (GRN) that encapsulate the gene interactions and pathways.

GRNs provide a level of resolution regarding the complex interactions of the transcriptome that has increased the predictive capability of RNASeq data. For example, computational algorithms have been developed to predict the interactions within the transcriptome. In predicting these interactions, the algorithms construct GRNs, where the nodes represent genes and edges between nodes represent the interaction between genes. The amount of information that can be provided in these nodes and edges is dependent on the algorithm. But generally, qualities like the number of edges starting at or ending at a certain node, directionality of edges, and weighting of the edges allow for these algorithms to not just put together a topological view of the gene interactions but provide predictive information regarding the order and importance of each interaction within the system.

Yet, while GRN techniques show great promise, in practice they have many limitations, including false positives, the inability to identify feedback loops, the inability to determine direct or indirect interactions, and the fact that current GRN algorithms require intensive computational power. Thus, while RNASeq can be used to examine other biochemical and high-throughput assays to combine gene expression data with things like cellular metabolome, proteome, and epigenome, conventional GRN techniques cannot take relative mRNA levels and sufficiently extrapolate all the interactions occurring between genes and gene products. Conventional GRN techniques are simply incapable of generating a full, accurate transcriptomic signature of a cell.

Conventional GRN techniques are data driven methods used to generate a network with weighted edges by estimating gene interactions directly from the transcriptome data. These methods are usually simple to implement, computationally efficient, and have proven to be accurate and effective. Example data driven methods include Correlation Networks, Information Theoretic Scores, and Regression-based methods.

Correlation Networks are simple to implement and can be easily deployed on genome-wide studies with very high numbers of experiments. However, there are limitations. High correlation does not mean the genes directly interact. Also, Correlation Networks are undirected, meaning the edges do not show the direction of the interaction/regulation. The WGCNA (weighted gene coexpression network analysis) is a commonly used Correlation Network algorithm.

Information Theoretic Scores methods can accommodate more subtle discrepancies than these Correlation Networks and represent the most common method for GRN inference. The Information Theoretic Scores method can scale to genome-wide networks, even if slightly more computationally intensive. However, these networks are still undirected and do not allow for a way of predicting behavior of a GRN. Common example algorithms are ARACNE, CLR, and MRNET.

Regression-based methods are typically more computationally intensive compared to the foregoing but provide directed networks allowing for predictive capabilities and are better at detecting indirect interactions between genes. However, the available RNASeq datasets for these indirect interactions are often smaller, leaving many of these methods more prone to error. Example algorithms include TIGRESS and GENIE3.

In contrast to these three data driven methods, probabilistic models have been proposed based on Gaussian graphical methods and Bayesian networks. Gaussian graphical methods treat gene expression measurements as a multivariate normal random vector (each entry of the vector representing the expression of one gene), and then estimate a precision matrix from multiple conditions using maximum likelihood estimation (MLE) for GRN inference. These methods have limitations, including difficulty when analyzing limited datasets and imply linearity in the relationship between genes even when the interactions between genes are not linear. Further, Gaussian graphical methods can also be computationally intensive. Bayesian networks also utilize MLE for GRN inference.

However, these can be computationally expensive, are less accurate on large datasets, and are unable to detect feedback loops in gene interactions.

Other conventional GRN methods fall under the category of dynamical models. These models allow for GRN inference under different conditions and include dynamic Bayesian networks, e.g., algorithms that circumvent the issue of feedback loops that cannot be detected using normal Bayesian Networks. While robust, dynamic Bayesian networks can be extremely costly to implement due to the intensive computational power needed.

Lastly, GRN methods have been proposed using deep-learning neural networks, built on machine learning. The use of machine learning is still very new in GRN inference, but several papers have been published in this field. These include: Ma A, et al. (2022, bioRxiv). DeepMAPS: Single-cell biological network inference using heterogeneous graph transformer Maseda F, et al. (2021, Front Genet.). DEEPsc: A Deep Learning-Based Map Connecting Single-Cell Transcriptomics and Spatial Imaging Data; Kishan KC, et al. (2019, BMC Systems Biology). GNE: a deep learning framework for gene network inference by aggregating biological information; and Chen J, et al (2021, Briefings in Bioinformatics). DeepDRIM: a deep neural network to reconstruct cell-type-specific gene regulatory network using single-cell RNA-seq data.

In short, there remains a desire for more efficient and more accurate techniques for generating gene regulatory networks and more broadly for determining spatial and temporal variations in gene networks.

### SUMMARY

The present application describes systems and methods for generating regulatory networks, such as spatial, and spatio-temporal networks, from structured-omic data, such as spatial or spatio-temporal gene expression data. For example, systems and methods are described for inferring cluster-specific gene regulatory networks from spatial gene expression profile data for patients. With the present techniques, spatially-varying Gaussian Markov random fields (SV-GMRF) and other techniques may be used to generate a network of sparse, context-specific clusters representing network relationships between genes. In these ways, the present techniques may generate gene regulatory networks from spatially-resolved transcriptomics datasets thereby identifying various types of relationships for the diagnosis and treatment of patient pathologies. The techniques herein are able to generate regulatory networks on drug-gene interactions, immunotherapy biomarker up regulation and down regulation, combinational immunotherapy relationships to transcriptome, patient specific tumor heterogeneity relationships, transcription factor and regulation of hub genes, and identifying combinatorial treatment/therapeutic regimens based on drug activity and response signatures.

In various examples, the present techniques deploy an efficient optimization problem that provides strong statistical and computational guarantees and works in lieu of conventional maximum likelihood estimation (MLE). The optimizations, for example, we show we can solve instances of SV-GMRFs with more than 2 million variables in less than 2 minutes. Indeed, we show examples herein where we apply the present techniques to study how gene regulatory networks in Glioblastoma are spatially rewired within tissue, and from that we identify prominent, heretofore unknown, activity of the transcription factor HES4 and ribosomal proteins as characterizing the gene expression network in the tumor pen-vascular niche that is known to harbor treatment resistant stem cells. Spatial network relationships have the potential to illuminate biologically synergistic mechanisms that drive disease at various regions within the tissue.

In accordance with an example, a method of inferring a regulatory network of interactions of regulators in structured-omic data, the method includes: obtaining, at one or more processors, the structured-omic data for a sample, the structured-omic data comprising expression data and corresponding location data; performing, at the one or more processors, a clustering process on the structured-omic data to generate a plurality of candidate clusters, each candidate cluster corresponding to a different regulatory network of a subpopulation in the structured-omic data, each candidate cluster comprising a plurality of nodes and edges between nodes, each node corresponding to a regulator and each edge corresponding to an interaction between regulators connected by each edge, for each candidate cluster, generating a precision matrix, at the one or more processors, establishing a plurality of candidate precision matrices; providing, at the one or more processors, the candidate precision matrices to a regularization stage, and in the regularization stage comparing the candidate precision matrices using an optimization that infers one or more output precision matrices each output precision matrix corresponding to different subpopulations in the structured-omic data, wherein the regularization stage is tunable to one or more regularization processes defining the optimization and tunable to select a weighting factor imposing a similarity between candidate precise matrices that is applied during the optimization; and converting, at the one or more processors, the one or more output precision matrices to one or more output inferred regulatory networks.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic diagram of a system for assessing a biological network of interactions to generate regulatory networks, in accordance with an example.

FIG. 2 is a schematic diagram of an example biological network computing device of the system of FIG. 1, in accordance with an example.

FIGS. 3A-3D illustrate an example operation of the biologic regulatory network platform and report generator. FIG. 3A illustrates operation of a gene regulatory network module and gene regulatory network informed treatment prediction module, as may be implemented by the system of FIG. 1, in accordance with an example. FIG. 3B illustrates resulting spatial transcriptomic (cluster) data, in accordance with an example. FIG. 3C illustrates a gene regulatory network, formed of five separate networks, as generated from a gene regulatory network inference process being performed on spatial transcriptomic data, in accordance with an example. FIG. 3D illustrates an optimized gene regulatory network with five optimized networks as performed by inference and optimization module of the biological network computing device of FIG. 2, in accordance with an example.

FIG. 4 illustrates the architecture of a biologic regulator network platform and report generator as may be implemented in by the biological network computing device of FIG. 2, in accordance with an example.

FIG. 5 illustrates an process for generating optimized inferred networks as may be performed by a biologic regulatory network platform of the biological network computing device of FIG. 2, in accordance with an example.

FIGS. 6A and 6B illustrate precision matrices for two adjacent clusters, with a parent cluster in FIG. 6A and a child cluster in FIG. 6B, in accordance with an example.

FIGS. 7A-7C illustrate plots of performance of different estimation methods for comparison, in accordance with an example.

FIG. 8A-8D illustrate plots of the Precision, Recall, and F1-score, as well as the runtime, respectively, of example processes herein, in accordance with an example.

FIGS. 9A-9D illustrate plot of the Precision, Recall, and F1 score of example processes herein, in accordance with an example.

FIG. 10, illustrates cluster data for different patient samples, in accordance with an example.

FIG. 11 illustrates deconvolution results of the cluster data of FIG. 10, in accordance with an example.

FIGS. 12A and 12B provide different illustrates of a sample tissue section showing large variations in number of expressed genes between clusters, in accordance with an example.

FIG. 13 illustrates five inferred gene regulatory networks generated by the biological regulatory network platform implementing processes herein, in accordance with an example.

FIG. 14 illustrates five inferred gene regulatory networks with particular regulatory interactions involving transcription factors highlighted, in accordance with an example.

FIG. 15 illustrates a gene ontology enrichment analysis report with the cluster-specific hub genes shown, in accordance with an example.

FIG. 16 illustrates another example architecture for implementing the processes herein, in accordance with an example.

### DETAILED DESCRIPTION

The present application presents systems and methods that infer regulatory networks from profiling data using novel, tunable, and adaptable regularization processes. The present techniques may be used, for example, to infer biological networks by performing regularization processes on spatially diverse and/or temporally diverse profile data, such as from structured-omic data collected from a subject. These biological networks, once inferred, can be used for biological hypothesis discovery of disease progression and treatment and for clinical decision making.

Structured-omic data includes regular structured data domains such as spatial-omic, temporal-omic, lattice-based data. Structured-omic data also includes irregular domains such as graph-informed domains and knowledge-informed domains. The techniques herein, including the regularization techniques, can be used to enforce sparsity and structure along any of those lines. In some examples, structured-omic data includes any structure based-omic data, including generated omic data, such as omic data generated using deep learning models such as transformer models of generative AI. Example spatial-omic data includes “spatial transcriptomic data,” which refers expression data captured for different locations within a tissue sample, where that data is coded or in some other way associated with the spatial locations at which it was captured in the sample. Indeed, references herein to “spatial transcriptomic” data are intended to include spatial-omic data more broadly, and in some examples, structured-omic data more broadly still.

The structured-omic data may be “temporal transcriptomic data” coded with temporal data. Use of the term “structured-omics” herein is intended to include one or more of spatial-omics, termporal-omics, or other structure based-omics. Examples of structured-omics herein include RNASeq data, spatially encoded RNASeq data, mRNA data, DNA data, miRNA, epigenomics, proteomics, or integrated modalities between these -omic platforms, like integrated spatial transcriptomic-epigenomics data, integrated transcriptomic-proteomics, integrated multiplexed IF-transcriptomics, spatial Clustered Regularly Interspaced Short Palindromic Repeats (CRISPR)/CRISPR Associated (CAS) screens. This spatial transcriptomic data may represent different sub-populations of data corresponding to different locations in a tissue sample or across multiple tissue samples. The spatial transcriptomic data may represent different sub-populations of data obtained at different times providing expression data corresponding to different points in time (temporally different). Techniques herein analyze such structured-omic data to infer spatial, temporal, and/or spatio-temporal interactions between cells, genes, etc., through a regularization framework analyzing structured-omic data.

More broadly, however, the present techniques may be implemented as any suitable structured-omic platform. With the present techniques, systems and methods may receive any number of different profiling data sets, biological or non-biological, and infer regulatory networks from the profiling data through appropriately adapted regularization frameworks.

In various examples, regularization frameworks used herein to infer regulator networks. They perform optimizations using different informed regularization processes. Informed regularization processes include, by way of example, domain-informed, knowledge-informed, graph-informed, platform-informed, structure-informed, and physics-informed. These may represent different axes along which the techniques herein can enforce similarity within a dataset, i.e., a set of related time instants, a subregion of pixels/voxels in space, a domain within which the gene dynamics (or network dynamics) is expected to be similar—like within a subset of measurement platforms, or a graph based on knowledge of molecular (like gene-gene) interactions. For example, in various examples, biological networks that vary in space and/or time can be inferred using regularization frameworks that have domain informed processes that include sparsely-changing optimizations and smoothly-changing optimizations. From there, assessment can be made of multiple biological hypotheses and multiple different potential treatments from the same inferred biological network(s). The ability to assess additional phenomenological insight into the interpretation of input profiling data based on pseudo-dynamizing static datasets and the ability to assess longitudinaVdynamically-acquired datasets, allows for invaluably efficient and faster mechanisms to assess the transcriptome in general and pathologies more particularly, including spatio-temporal transitions in cell state and local environments in tissue.

As noted, in addition to inferring regulatory networks on interactions between cells, genes, etc. from spatially and/or temporally varying profiling data, the present techniques can be extended to other dimensions (beyond space and time) to infer interactions. The techniques, for example, can be applied where proteomics and transcriptomic data are available for the same specimen (e.g., with cellular indexing of transcriptomes and epitopes sequence data, termed CITE-seq data) to infer regulatory protein networks. Indeed, the present techniques are generalizable to be applicable to a variety of applications where sequencing/omics data is available for analysis. These include, but are not limited to, other panomic characteristics such as “genomics, epigenomics, chromatin state, proteomics, metabolomics, transcriptomics, etc., and sociological and environmental characteristics of a patient in near real-time.”

In various examples, the present techniques use structured-omic data in the form of transcriptomic data to generate novel gene regulatory networks (GRNs) that encapsulate gene interactions and pathways. GRNs are networks in which nodes represent genes and edges between nodes represent the interaction between genes. As such, GRNs may be used to examine the complex interactions of the transcriptome, for example, interactions in response to potential treatments. The amount of information provided in the nodes and edges of a GRN is dependent on the algorithm used to generate the GRN. Generally, qualities like the number of edges starting at or ending at a certain node, the directionality of edges, and the weighting of the edges allow GRNs to provide a topological view of gene interactions and to provide predictive information regarding the order and importance of each interaction within a system. Yet, as discussed above, there are many limitations in current GRN techniques, including the generation of false positive edges between genes, the inability to identify feedback loops between interactions, and the inability to determine direct or indirect interactions. Also, current GRN algorithms require intensive computational power making them time consuming and costly.

The techniques herein include various methods that overcome these and other limitations in GRN technology in particular, and in spatial transcriptomics more generally. For example, techniques are described that perform RNASeq analyses for transcriptomics applications using efficient and optimized spatially-varying Gaussian Markov Random Fields (SV-GMRF). Generally speaking, SV-GMRF is used as a model for genes in a transcriptome, to provide GRN inference, under structural constraints like spatial/temporaVspatio-temporal variation. Most Gaussian models rely on maximum likelihood estimations (MLE) for GRN inference. However, as discussed above, conventional use of MLE leads to high computational costs especially when scaling to larger RNASeq datasets. The present techniques overcome these cost and speed limitations by providing regulatory network (e.g., GRN and others) inference processes that are computationally much more efficient (e.g., optimization methods in lieu of MLE). For example, GRN inference techniques are provided that include Backward Mapping Deviation optimizations, Absolute Regularization optimizations, and/or Spatial Regularization optimizations. As discussed further, using an example of a GRN inference model in accordance with the present techniques, more than 2 million SV-GMRFs were resolved in approximately 2 minutes. The model was then applied to RNASeq datasets of glioblastoma samples to determine how GRNs are spatially rewired during disease progression, that is, resulting in both spatially-derived and temporally-derived transcriptome changes. Indeed, the present techniques were able to identify the transcription factor HES4 and ribosomal proteins as important drivers of tumor adaptation in the pen-vascular niche of glioblastoma. As demonstrated, the techniques provide exciting and new insight into GRNs that allow for predictions to be made regarding possible therapeutic targets and other diagnostic outcomes.

In accordance with an example, the techniques herein include systems and methods of inferring a regulatory network of interactions of regulators in structured-omic data. For example, the methods include obtaining the structured-omic data for a sample, the structured-omic data comprising expression data and corresponding location data, and performing a clustering process on the structured-omic data to generate a plurality of candidate clusters, each candidate cluster corresponding to a different regulatory network of a subpopulation in the structured-omic data, each candidate cluster comprising a plurality of nodes and edges between nodes, each node corresponding to a regulator and each edge corresponding to an interaction between regulators connected by each edge. The methods further include for each candidate cluster, generating a precision matrix thereby establishing a plurality of candidate precision matrices. The methods further include providing the candidate precision matrices to a regularization stage, and in the regularization stage comparing the candidate precision matrices using an optimization that infers the output precision matrices. Each output precision matrix corresponds to different subpopulations in the structured-omic data, wherein structured-omic similarities and dependencies are discovered and promoted via the regularization. The regularization stage is tunable to one or more regularization processes defining the optimization and tunable to select a weighting factor imposing a similarity between candidate precise matrices that is applied during the optimization.

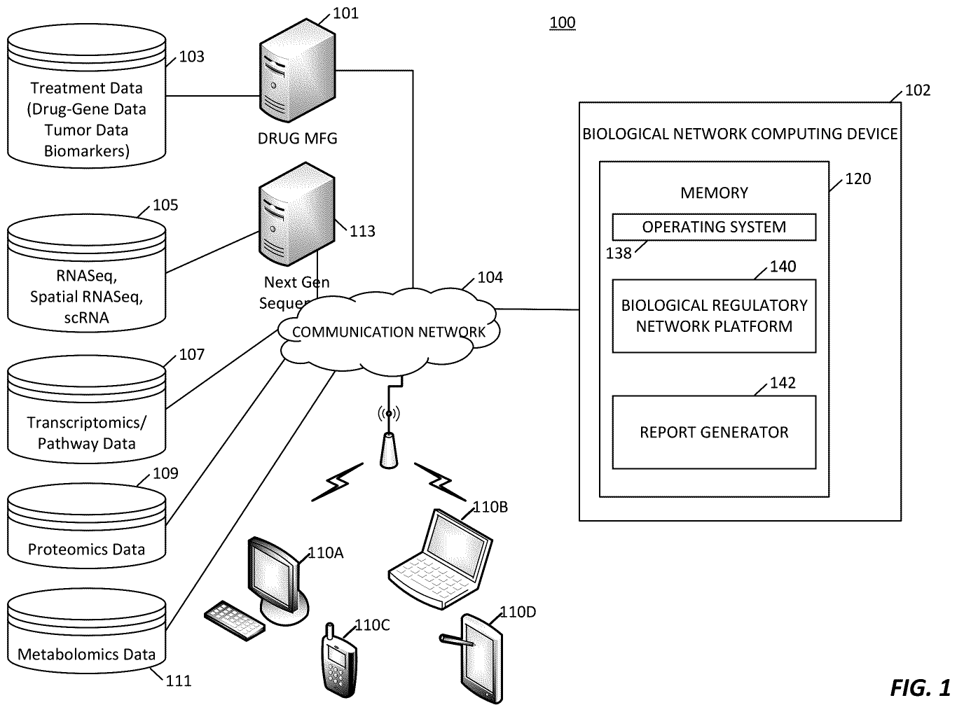

FIG. 1 illustrates a system architecture 100 for assessing a biological network of interactions to generate regulatory networks, such as spatial networks, temporal networks, and spatio-temporal networks, from structured-omic data, such as gene expression data. The system architecture 100 may be used to generate regulatory networks of any number of interconnected variables defining a system, where those variables interact according to some underlying and hidden structure of that system, and where spatial data is available or can be generated. Examples of such systems include brain connectivity and operation, financial markets, and genelmolecular networks.

In the illustrated example, the system architecture 100 is described in reference to generating biological regularly networks, such as a gene regulatory networks (GRN). GRN's refer to the complex set of interactions between genes, transcription factors, and other molecules that control gene expression. These networks help to regulate the timing, location, and level of gene expression in cells and tissues. Generally, GRN's are a collection of molecular or biological regulators that interact with each other. The regulators can be DNA, RNA, protein or any combination of these forming a complex, such as a specific sequence of DNA and a transcription factor to activate that sequence. The interaction can be direct or indirect (through transcribed RNA or translated protein). Some regulators with the same genes or variables from different places interact with each other to various degrees. In some examples, we want to infer all these networks while utilizing their similarity.

In the illustrated example, the system architecture 100 includes a biological network computing device 102 communicatively coupled to different data sources providing structured-omic data, such as transcriptomic data and other data (including but not limited to other omic data sources and clinical data). The biological network computing device 102 is coupled to a communication network 104 that is further communicatively coupled to different data sources, including a treatment data store 103 accessible via a server 101. As used herein, “treatment data” may be any type of data corresponding to responsiveness to cancer treatment efficacy, including, for example, known drug-gene interaction data, tumor responsive data, known radiation sensitivity biomarkers, immunotherapy biomarkers, combinational therapy biomarkers, drug sensitivity markers or signatures, demographic data, etc. The system 101 may be a health care system providing access to electronic health records, a drug manufacturer providing access to drug responsiveness data, or other suitable computing system 101.

Gene expression data in the form of RNA sequencing data (RNASeq), spatial RNASeq, single cell RNASeq (scRNA) data is accessible through data store 105, for example a data store associated with next generation sequencing computer system 113. Transcriptomic data, such as gene pathway data is accessible through a data store 107. Proteomics data is accessible through a data store 109. Metabolomics data is accessible through a data store 111. More generally, the biological network computing device 102 may be connected to any number of panomic data, whereas used herein, the term “panomics” may refer to a range of molecular biology technologies/platforms related to the interaction of biological functions within a cell and with other functions within the human body. For example, panomics may include genomics, epigenomics, chromatin state, transcriptomics, proteomics, metabolomics, biological networks and systems models, etc. Panomic data may be specific to various point in time and to specific tissues and linages of cells, so that panomic data collection is connected to these features and may also be collected and used for a plurality of tissues, lineages, and temporal points connected to phenotypes of interest for a patient. A patient's panomics may relate to biomarkers for multiple phenotypes such as pharmacologic responses to drugs, disease risks, comorbidities, substance abuse problems, etc. Panomics data may be generated and collected for the purpose of a specific set of medical decisions at a discrete point in time, and also may be harvested from the sum record of previously collected panomics data at points in the past for an individual patient.

The biological network computing device 102 may be further connected to various personalized devices also connected to the network 104, through which health care professionals and/or patients can enter identification data and access regulatory network reports generated by the computing device 102. The devices may present an instantiation of an accessing (app) to provide such identification data and to allow the devices to be individually authenticated from communication with the computing device 102. In the illustrated examples, individual devices include a computing terminal 110A, a laptop computer 110B, a mobile cellular device 110C, and a mobile tablet computing device 110D, each available to a different healthcare professional and/or patient. Other patient devices include personal mobile and/or monitoring devices, such as smart watches and other wearable monitoring devices such as heart rate monitors and heath things. Of course, provided devices may be coupled to the computing device 102 through the network 104 and these provided devices may be computer terminals, laptop computers, mobile cellular devices, mobile tables, and the like, as well.

In the example of FIG. 1, the system 100 is implemented on a single network accessible server implementing the biological network computing device 102. In other examples, the functions of the biological network computing device 102 may be implemented across distributed devices connected to one another through a communication link. In other examples, functionality of biological network computing device 102 may be distributed across any number of devices, including the portable personal computer, smart phone, electronic document, tablet, desktop personal computer devices shown, and personal mobile and/or monitoring devices, such as wearable devices like smart watches, health rings, and heart rate monitors. In other examples, the functions of the biological network computing device 102 may be cloud-based, such as, for example one or more connected cloud CPU(s) customized to perform machine learning processes and computational techniques herein.

The network 104 may be a public network such as the Internet, private network such as research institution's or corporation's private network, or any combination thereof. Networks can include local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), Bluetooth, Bluetooth Low Energy, AirPlay, or other types of protocols. Moreover, the network 104 can include a number of devices that facilitate network communications and/or form a hardware basis for the networks, such as switches, routers, gateways, access points (such as a wireless access point as shown), firewalls, base stations, repeaters, backbone devices, etc.

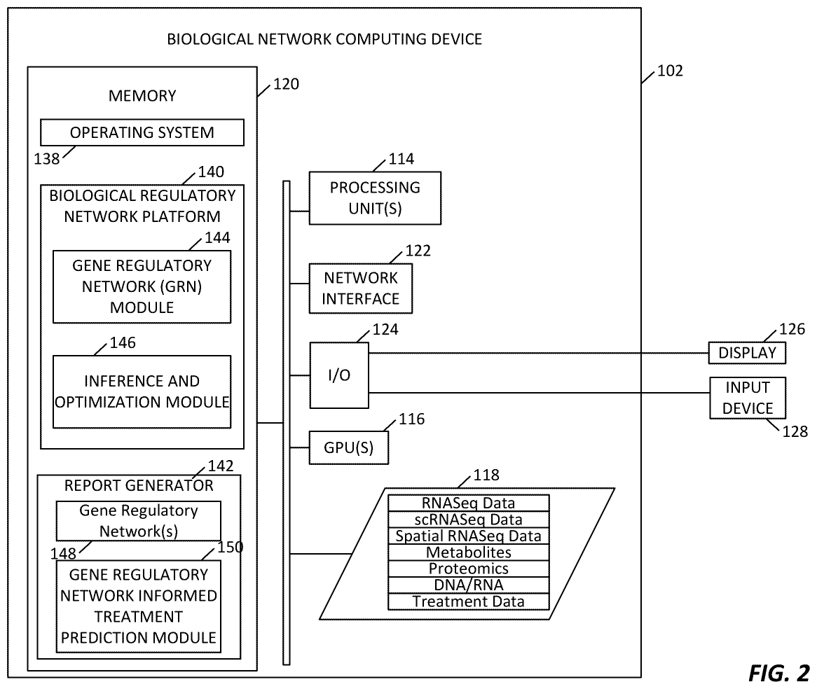

As shown in FIG. 2, the biological network computing device 102 includes one or more processing units 114, one or more optional graphics processing units 116, a local database 118, the computer-readable memory 120, a network interface 122, and Input/Output (I/O) interfaces 124 connecting the computing device 102 to a display 126 and user input device 128.

The computer-readable media 120 may include executable computer-readable code stored thereon for programming a computer (e.g., comprising a processor(s) and GPU(s)) to the techniques herein. Examples of such computer-readable storage media include a hard disk, a solid state storage device/media, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 102 may represent a CPU-type processing unit, a GPU-type processing unit, a field-programmable gate array (FPGA), another class of digital signal processor (DSP), or other hardware logic components that can be driven by a CPU.

In the illustrated example, the database 118 includes panomic data, for example, stored in any of the data stores 105, 107, 109, and 111, whether specific to a particular patient, a population of patients, reference databases, etc. In the illustrated example, the database 118 stores RNASeq data, scRNASeq data, spatial RNASeq data, metabolite data, proteomic data, and DNA/RNA data. Further treatment data from the data store 103 may be stored in the database 118.

In the illustrated example, the biological network computing device 102 includes a computer-readable memory 120 storing an operating system 138, a biological regulatory network generation platform 140 and a report generator 142 both configured to execute various processes described and illustrated herein. The biological regulatory network generation platform 140 includes a gene regulatory network (GRN) module 144 that is configured to generate GRNs (or other regulatory networks) in response to receiving spatial transcriptomic data. An inference and optimization module 146, within the platform 140, is configured to perform an optimization on generated GRN(s), in accordance with the techniques herein. These optimizations may be nonlinear and nonconvex, and in some examples contain one or both of a sparsely-changing regularization process and a smoothly-changing regularization process. The result may be a set of optimized GRNs or other regulatory networks, for example, that have been optimized to identify core transcription factors, hub genes, master regulators, druggable nodes, intervenable/perturbable nodes of the GRN.

The report generator 142 stores optimized GRN(s) 148 from the platform 140. The report generator 142, in some examples, includes a gene regulatory network informed treatment prediction module 150 configured to identify, from the optimized GRN(s) 148, a subset of available treatments that correspond to the identified nodes and/or edges in different microenvironments of the tissue, and in some examples a treatment plan thereof.

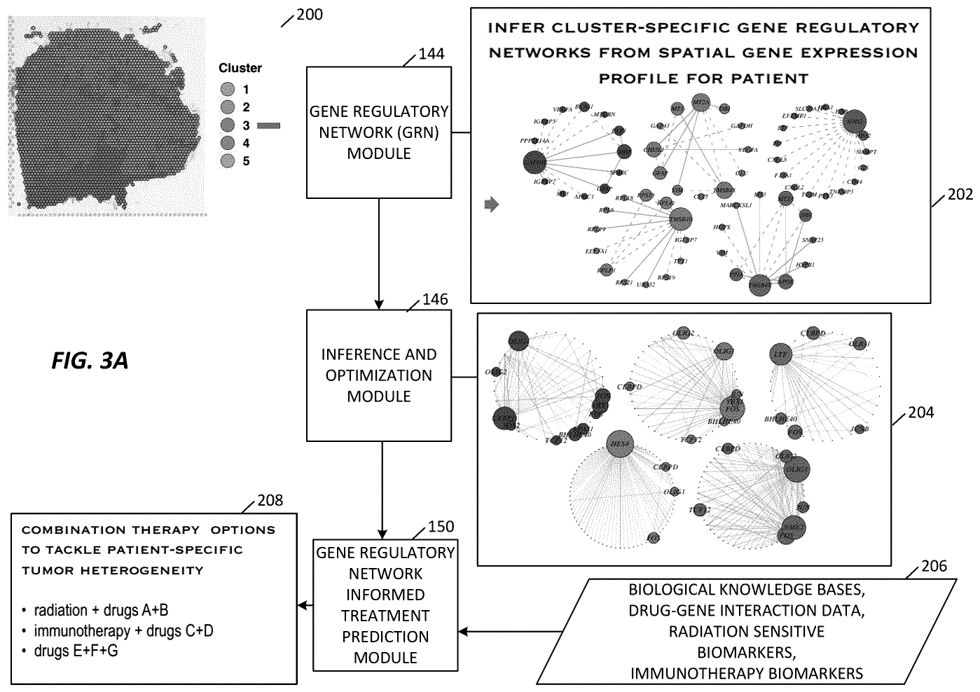

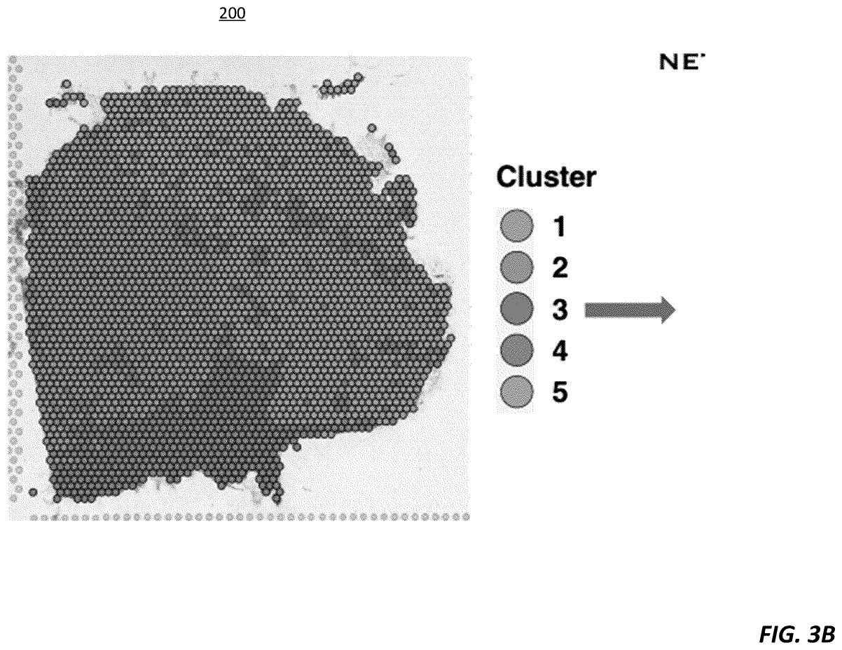

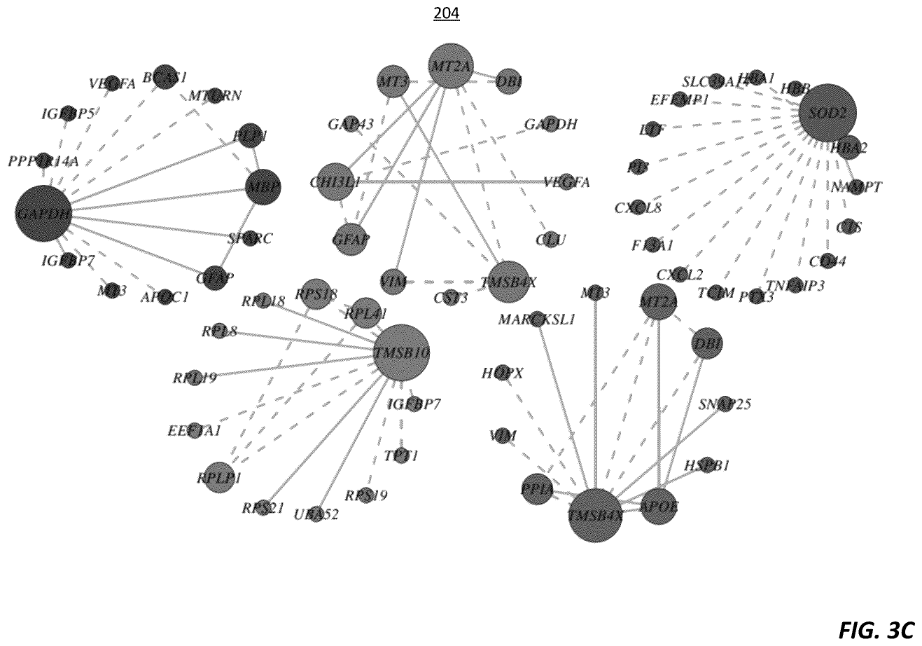

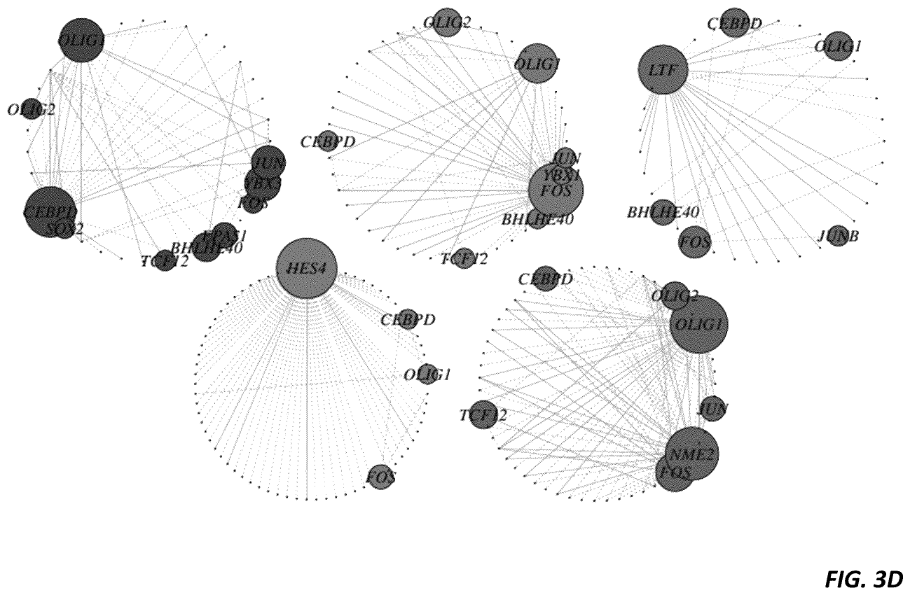

FIGS. 3A-3D illustrate an example operation of the biologic regulatory network platform 140 and report generator 142. Spatial-omic data, in this example, spatial transcriptomic data 200 (e.g., from the data store 105, and also shown in FIG. 3B), is received at the gene regulator network module 142. In the illustrated example, the spatial transcriptomic data 200 is provided as pre-clustered data, with the data having already gone through a clustering process. Clustering herein includes techniques for partitioning samples based on distance between points, such as a geometrical distance. By way of example, the data has been clustered in five different groups, which generally, although not entirely, each correspond to a different spatial region of a sequenced tissue sample. In the illustrated example, the GRN module 144 has been configured to perform a gene regulatory network inference process on the spatial transcriptomic data 200, generating a GRN 202 (also shown in FIG. 3C), formed of five separate networks, each corresponding to a spatio-temporally (or otherwise structurally coherent) partition of the overall tissue. The GRN module 144 may be configured to infer cluster-specific GRN(s) using techniques, such as those discussed herein. The GRN module 144 sends the generated GRN(s) 202 to the inference and optimization module 146 configured, in accordance with the techniques herein, to perform optimizations to generate optimized spatial GRNS, spatio-temporal GRNs, or other regulatory networks. In the illustrated example, the inference and optimization module 146 generates an optimized GRN 204 (also shown in FIG. 3D) with five separate optimized networks, each corresponding to one of the networks in the GRN 202, although in other examples, the optimized regulator network 204 may be formed of other networks entirely resulting from the processes herein. More particularly, in the illustrated example, each of the five networks has been optimized to show a dominant transcription factor (or master regulator set) for each network. For example, the GRNS of FIGS. 3A-3D indicate that the present techniques identified dominant roles played by OLIG1/2 and CEBPD in tumor adaptation across tissue regions of a sample. These are known master regulators of Glioblastoma. In addition to these factors, the present techniques identified regional TFs such as LTF and HES4, as playing an important role in tumor adaptation to the specific microenvironment of the sample. Through the generated GRN reports, clinicians can visually see the degree of heterogeneity of tumor states within this 6.5×6.5 mm section of tissue and identify specific biological actors that must be synergistically targeted in order to treat the disease.

While the inference and optimization module 146 is shown separately from the GRN module 144, in some examples, all or part of the operations of each may be combined into a single module, i.e., a single set of computer executable instructions.

In FIG. 3A, the optimized GRN 204 is received at the report generator 142, where in some examples, the gene regulatory network informed treatment prediction module 150 compares the optimized regulatory network data to data of biological knowledge bases 206. Such comparison data may be drug-gene interaction data, radiation sensitive biomarker data, immunotherapy biomarker data, and drug sensitivity -omics signatures. The module 150 is configured to assess the received data sets and generate a report 208, which may contain combination therapy options that have a higher likelihood of tackling a patient-specific tumor heterogeneity. That report 208 may contain data (such as percentage of match to the patient) on radiation therapy and combination drugs A+B, on immunotherapy and combination drugs C+D, and a multifactorial (e.g. three-drug) combination of drugs E+F+G. The drug lettering is merely provided by way of example. The report 208 may reflect the prediction module 150 having identified optimum combinations of drugs (from a large data set of 100s to 1000s of possible drugs) for reach of the cancer treatment types: radiation, immunotherapy, drug therapy.

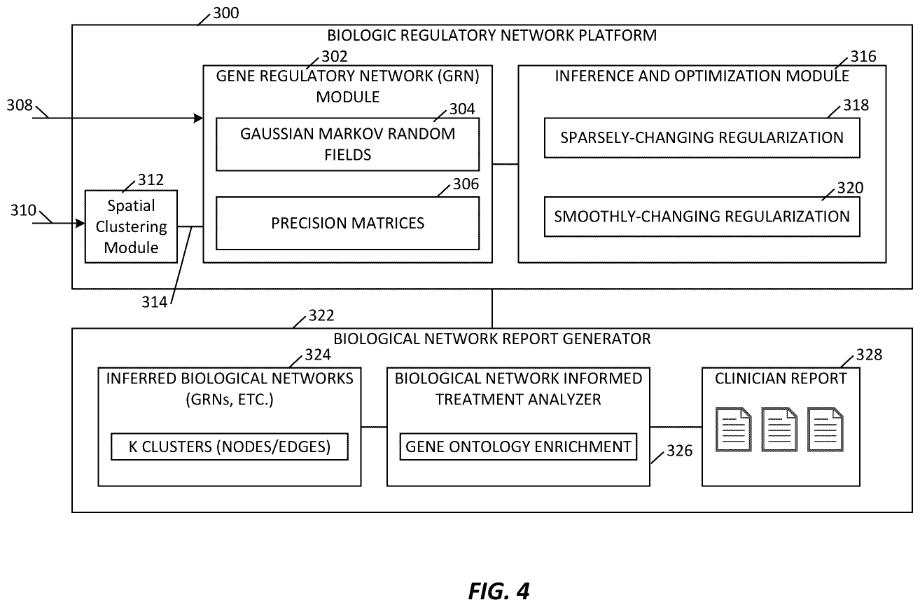

FIG. 4 illustrates an architecture 300 of a biologic regulator network platform and report generator as may be implemented in an example of the biological network computing device 102 of FIG. 2. In the illustrated example, a regulatory network module 302 contains a gene regulatory network (GRN) generator in the form of a Gaussian Markov random fields generator 304. As such, the regulatory network module 302 may be considered a gene regulatory network (GRN) module in the illustrated example.

A Gaussian Markov Random Field (GMRF) is a statistical model that describes a set of random variables that are related to each other through a Gaussian distribution and a Markov property. In a GMRF, each random variable represents a node in a graph, and the edges between nodes represent the relationships between the variables. The Gaussian distribution specifies the distribution of the random variables, and the Markov property specifies that the conditional distribution of each variable given its neighbors is Gaussian. In other words, a GMRF is a probabilistic graphical model in which the joint probability distribution over the variables can be decomposed into a product of conditional probabilities, with each variable conditioned only on its neighbors in the graph. This leads to efficient algorithms for inference and learning in GMRFs, such as the widely-used Gaussian belief propagation algorithm. While the regulatory network module 302 includes the GMRF generator 304, the regulatory network platform 300 may be configured to perform any of a variety of different mappings of structured-omic data, including integrative probabilistic reasoning of omic modalities like epigenomics, chromatin maps, protein interactions etc. Example mappings for generating visualizable connections of network complexity include network graphs, heat maps, circos plots, and Sankey diagrams.

To provide a computationally efficient way to represent the regulatory networks (e.g., GMRFs) created by the generator 304, the regulatory module 302 further includes precision matrices 306. A precision matrix (also known as the concentration matrix or the inverse covariance matrix) is a matrix that encodes the conditional independence relationships between the random variables in the model. Specifically, the precision matrix is defined as the inverse of the covariance matrix of the random variables. In other words, if we have a GMRF with n random variables, then the precision matrix P is an n×n matrix where P ij=0 if variables i and j are conditionally independent given all other variables, and P ij is nonzero if variables i and j are conditionally dependent given all other variables. Moreover, the nonzero entries of the precision matrix reflect the strength of the conditional dependencies between the variables. The precision matrices 306 allow for efficient computation of the joint probability distribution of the random variables, as well as efficient inference and optimization. Further, one of the advantages of using the precision matrices 306 to represent the conditional dependencies in a generated GMRF is that it leads to sparsity in the matrix, since many of the entries are zero. This sparsity allows for efficient computation and storage of the matrix, which is particularly important for high-dimensional models.

In operation, the GRN module 302 may receive data that has already been encoded with spatial information, such as structured-omic data 308. Or in other examples, expression data 310 may be provided to a spatial clustering module 312 that generates encoded structured-omic data 314 (such as transcriptomic data) sent to the GRN module 302.

In some examples, the GRN module 302 is an example regulatory network that receives structured-omic data and performs a clustering process on that structured-omic data to generate a plurality of candidate clusters that may each correspond to a different regulatory network of a subpopulation in the structured-omic data. These clusters (or different regulatory networks) are each converted to a precision matrix, thus providing a domain for inference and optimization in accordance with techniques herein. These generate precision matrices that are consumed by an inference and optimization module that contains a regularization stage that compares the precision matrices using an optimization process to infer one or more optimized precision matrices that correspond to different subpopulations in the structured-omic data. This regularization stage may be tunable to one or more regularization processes defining the optimization and tunable to select a weighting factor imposing a similarity between candidate precise matrices that is applied during the optimization.

In the illustrated example, an inference and optimization module 316 contains two different regularization processes executed as software modules for optimizing the GRNs of the module 302, in particular, by being configured to operate on the precision matrices 306. The inference and optimization module 316 includes a sparsely-changing regularization module 318 and a smoothly-changing regularization module 320. These regularization modules 318 and 320 may implement optimization protocols described in various processes and methods described herein.

The example architecture 300 further includes a biological network report generator 322 that stores inferred biological networks 324, such as GRNs, etc. generated by the module 302. These inferred biological networks 324 may contain a plurality of optimized clusters 326 defined by nodes and edges, such as the GRN clusters 202 and the optimized GRN clusters 204 of FIG. 2.

To assess the inferred biological networks 324 against treatment options, the report generator 322 further includes a treatment analyzer 326. In an example, the treatment analyzer 326 may include any number of processes to analyze treatments based at least in part on the inferred biological networks. For example, the analyzer 326 may determine a percentage of similarity between historical network data (e.g., for successfully treated populations) and inferred biological networks. The analyzer 326 may determine a projected likelihood of treatment success based on the inferred biological networks. The analyzer 322 may identify a subset of possible treatment options based on the inferred biological networks. In an example, the analyzer 326 includes a gene ontology enrichment analyzer. Clinician reports 328 may include the inferred biological networks 324 and/or recommended therapy reports from the analyzer 326, gene ontology enrichment reports, etc.

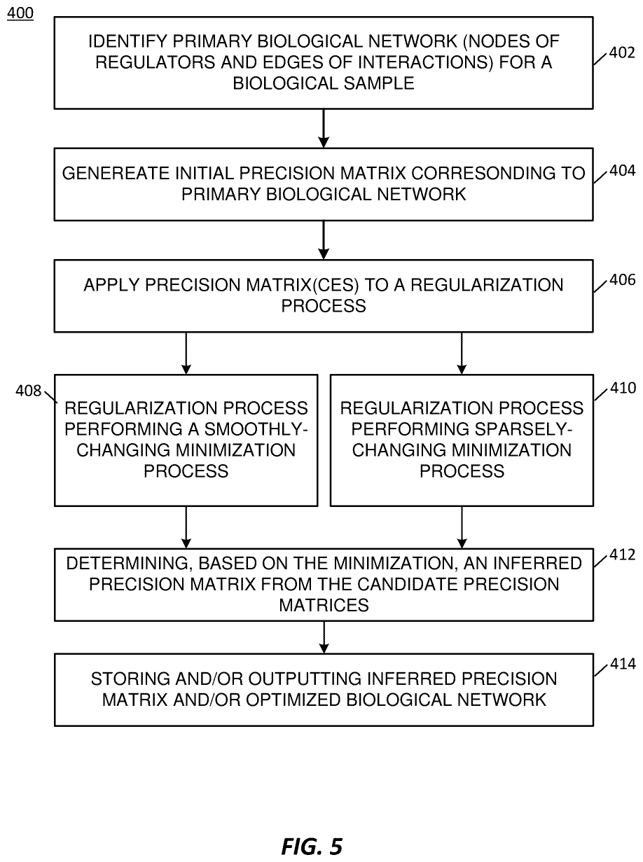

FIG. 5 illustrates an example process 400 for generating optimized inferred networks as may be performed by the biologic regulatory network platform 140 of the computing device 102 of FIG. 1 and/or the biologic regulatory network platform 300 of FIG. 3A. At block 402, spatial-omic data (such as spatial transcriptomic data or other spatially encoded data) from a biological sample is collected and an inference process is performed to generate an initial biological network, containing nodes of various regulators of a biological network and edges representing interactions between those regulators, such as for example a GMRF or other GRN. The biological network of block 402 may contain numerous networks, also termed clusters herein. At a block 404, a precision matrix is generated for the biological network, i.e., for each cluster in the biological network from the block 402. In some examples, the block 404 may perform an initial regularization process to estimate the precision matrix while avoiding overfitting. Overfitting can occur when the precision matrix is estimated directly from the data, leading to a complex and possibly overparameterized model that fits the training data well but generalizes poorly to new data. An example regularization that can be used to develop the precision matrix is a shrinkage regularization. Shrinkage regularization involves adding a penalty term to the likelihood function that encourages the precision matrix to be sparse, i.e., to have many zero entries. This penalty term can take different forms, such as the L1-norm or the L2-norm of the precision matrix.

To convert an initial precision matrix (or set of matrices) from block 406 into an optimized precision matrix (or matrices) of different regulator interactions, a block 408 performs a smoothly-changing minimization regularization process and a block 410 performs a sparsely-changing minimization regularization process. From these smoothly-changing and sparsely-changing minimizations, a block 412 determines an inferred precision matrix (or matrices) and these are stored at a block 414 or converted, at the block 412, into network mappings such as optimized GMRFs or other GRNs that are stored at the block 414.

An example operation of the process 400, the regularization minimization processes of blocks 408 and 410, and the inferred precision matrix process of block 412 is now described.

We now describe an example of the process 400.

### Example Technical Problem Formulation

In the following example, we formally defined the problem of inferring SV-MRFs using the present techniques, setting forth the following notations.

Notations: The i-th element of a vector v or vₜ is denoted as vᵢ or v_{t;i}. For a matrix M, the notations M_{i:} and M_{:j} denote the i-th row and j-th column, respectively. Moreover, for an index set S and a matrix M, the notations M_{S:} and M_{:S} refer to a submatrix of M with rows and columns indexed by S, respectively. For a matrix M or a vector v, the notations ∥M∥_{lq} and ∥ν∥_{q} correspond to the element-wise [symbol] -norm of M and [symbol] -norm of v, respectively. Moreover, ∥M∥_{q} and ∥M∥ₘₐₓ are the induced q-norm and the element with the largest absolute value of the matrix M, respectively. Moreover, ∥M∥₀ denotes the total number of nonzero elements in M. We use M>0 to show that M is positive definite. For a vector v and matrix M, the notations supp(v) and supp(M) are defined as the index sets of their nonzero elements. Given two sequences f(n) and g(n) indexed by n, the notation f(n)≤g(n) implies f(n)≤Cg(n) for some constant C<∞. Moreover, the notation f(n) [symbol] g(n) implies that f(n)≤g(n) and g(n)≤f(n). The sign function sign(·) is defined as sign(x)=x/|x| if x≠0 and sign(0)=0. Accordingly, when x is a vector, the function sign(x) is defined as [sign(x₁) sign(x₂) . . . sign(xₙ)]^{T}.

We considered data samples from K different Gaussian distributions with d×d covariance matrices Eₖ^{*}εS₊^{d}, k=1, . . . K and sparse precision matrices Θₖ^{*}=Eₖ^{*-1}, k=1, . . . , K. Let {xₗ^{k}}ᵢ₌₁^{nk} be nₖ independent samples, each with dimension d, drawn from the k-th distribution, i.e., xᵢ^{k}˜N(0, Σₖ^{*}), for every i=1, . . . , nₖ and k−1, . . . ,K. The zero-mean assumption on the distributions is without loss of generality and can be achieved by normalizing the data. Our goal was to estimate the precision matrices (Θₖ^{*})ₖ₌₁^{K} given the samples. The most commonly-used method to perform this task is via maximum likelihood estimation (MLE) with an [symbol] regularizer (also known as Graphical Lasso):

$${\hat{Θ}}_{k} = arg {min}_{{Θ}_{k} ≻ 0} Tr ( {Θ}_{k} {\hat{Σ}}_{k} ) - log det ( {Θ}_{k} ) + λ {‖ {Θ}_{k} ‖}_{{ℓ}_{1}}$$

where Tr(·) is the trace operator and

$${\hat{Σ}}_{k} := \frac{1}{{n}_{k}} {Σ}_{i = 1}^{{n}_{k}} {x}_{i}^{k} {x}_{i}^{{k}^{T}}$$

is the sample covariance matrix for distribution k. λ is a regularization coefficient that controls the level of sparsity in the inferred precision matrix. A major drawback of the above estimation method is that it ignores any common structure among different distributions. To address this issue, a common approach is to consider a joint estimation method (also known as joint Graphical Lasso):

$$\{ {\hat{Θ}}_{k} \} = arg {min}_{{Θ}_{k} ≻ 0} ( Tr ( {Θ}_{k} {\hat{Σ}}_{k} ) - log det ( {Θ}_{k} ) + λ {‖ {Θ}_{k} ‖}_{{ℓ}_{1}} ) + P ( {\{ {Θ}_{k} \}}_{k = 1}^{K} ) \qquad ( 1 )$$

where the term P((Θk)ₖ₌₁^{K}) is a penalty function that encourages similarity across different precision matrices. A major difficulty in solving joint Graphical Lasso, however, is its computational complexity: in order to obtain an E-accurate solution, typical numerical solvers for (1) have complexity ranging from [symbol] (Kd⁶ log (1/E)) (via general interior-point methods) to [symbol] (Kd³/∈) (via tailored first-order methods, such as ADMM). Solvers with such computational complexity fall short of any practical use in the large-scale settings. Indeed, the prohibitive worst-case complexity of methods based on Graphical Lasso is also exemplified in their practical performance.

### Example Proposed Operation

To address the aforementioned issues, we proposed the following surrogate optimization problem for estimating sparse precision matrices:

$${\{ {\hat{Θ}}_{k} \}}_{k = 1}^{K} = arg {min}_{{\{ {\hat{Θ}}_{k} \}}_{k = 1}^{K}} {\underbrace{{\sum }_{k = 1}^{K} ‖ {Θ}_{k} - {\tilde{F}}^{⋆} ( {\hat{Σ}}_{k} ) {‖}_{{l}_{2}}^{2}}}_{backward mapping deviation} + {\underbrace{μ {\sum }_{k = 1}^{K} ‖ {Θ}_{k} {‖}_{{l}_{1}}}}_{absolute regularization} + {\underbrace{γ {\sum }_{l > k} {W}_{kl} ‖ {Θ}_{k} - {Θ}_{l} {‖}_{{l}_{q}}^{q}}}_{spatial regularization} \qquad ( Elem - q )$$

In the above optimization, the backward mapping deviation expression captures the distance between the estimated precision matrix and the so-called approximate backward mapping, described herein. Moreover, the absolute regularization expression promotes sparsity in the estimated parameters, whereas the spatial regularization expression encourages common spatial similarities among different parameters. For any given pair (k, l), the weight Wₖₗ⁻¹ can be interpreted as the “distance” between the k-th and l-th MRFs. Accordingly, a large value for Wₖₗ encourages similarity between Θₖ and Θₗ. In an example, two spatial similarities were applied, sparsity and smoothness:

Smoothly-changing GMRF: In smoothly-changing GMRFs, the adjacent precision matrices vary gradually. In this setting, q=2 can be used as the spatial regularizer in Elem-q to promote the smoothness in the parameter differences.

Sparsely-changing GMRF: In sparsely-changing GMRFs, the adjacent precision matrices differ only in a few entries. In this setting, q=1 is a natural choice for the spatial regularizer in Elem-q since it promotes sparsity in the parameter differences.

More generally, the weighting factor, Wₖₗ represents the similarity between precision matrices irrespective of the domains of the precision matrices. The weighting factor encourages similarity between precision matrices of subpopulations that differ based on location within a sample (spatially different subpopulations), based on time such as subpopulations representing progression of a cancer over time (temporally different subpopulations), based on combinations of location and time differing subpopulations (spatially-temporally different subpopulations). The precision matrices may represent any number of different subpopulation domains, and the weighting factor can be used to encourage the similarity between them. Other examples may be domain informed regularization process 520, a platform informed regularization process 522, a structure informed regularization process 524, and a physics informed regularization process 526.

Gamma is a tuning parameter that controls the sparsity level of the inferred precision matrices. A large value for Gamma leads to sparser precision matrices, while a small value yields dense precision matrices with fewer zero elements. Most spatially-varying graphical models have sparse precision matrices. The correct sparsity level of the precision matrices can be recovered by fine-tuning Gamma.

### Example Approximate Backward Mapping

In this example, the proposed optimization problem was contingent upon the availability of an approximate backward mapping. For a GMRF, the backward mapping is defined as the inverse of the true covariance matrix, i.e., F*(Σₖ^{*})=Eₖ^{*-1}=Θₖ^{*}. Based on this definition, a natural surrogate for the backward mapping is F*(Σₖ)=Σₖ⁻¹, where Σₖ is the sample covariance matrix for distribution k. However, in the high-dimensional settings, the number of available samples is significantly smaller than the dimension, and as a result, the sample covariance matrix Σₖ is singular and non-invertible. To alleviate this issue, Yang et al. Advances in Neural Information Processing Systems, Vol. 27, Curran Associates, Inc. (2014) introduce anapproximation of the backward mapping based on soft-thresholding. Consider the operator ST_{ν}(M): [symbol] ^{d×d}→ [symbol] ^{d×d}, where ST_{ν}(M)ᵢⱼ=Mᵢⱼ−sign (Mᵢⱼ)min{|Mᵢⱼ|,ν} if i≠j, and ST_{ν}(M)ᵢⱼ=Mᵢⱼif i=j. Given this operator, the approximate backward mapping is defined as F*(Σₖ)=ST_{ν}(Σₖ)⁻¹, for every k=1, . . . ,K. An important property of this approximate backward mapping is that it is well-defined even in the high-dimensional setting nₖ<<d with an appropriate choice of the threshold v. Given this approximate precision matrices from Elem-q are close to their true counterparts with an appropriate choice of parameters.

### Example Decomposability

A property of the expression Elem-q is that it naturally decomposes over different coordinates of the precision matrices: for every (i, j) with 1≤i≤j≤d, the ij-th element of (Oₖ)ₖ₌₁^{K} can be obtained by solving the following subproblem:

$${\{ {\hat{Θ}}_{k ; ij} \}}_{k = 1}^{K} = arg {min}_{{\{ {Θ}_{k ; ij} \}}_{k = 1}^{K}} {\sum }_{k = 1}^{K} {( {Θ}_{k ; ij} - {[ {\tilde{F}}^{⋆} ( {\hat{Σ}}_{k} ) ]}_{ij} )}^{2} + μ {\sum }_{k = 1}^{K} ‖ {Θ}_{k ; ij} ‖ + γ {\sum }_{l > k} {W}_{kl} \vert {Θ}_{k ; ij} - {Θ}_{l ; ij} {\vert}^{q} , \qquad ( Elem - ( i , j , q ) )$$

Recall that the original problem Elem-q has Kd(d+1)/2 variables. The above decomposition implies that Elem-q can be decomposed into d(d+1)/2 smaller subproblems, each with only K variables that can be solved independently in parallel. This is in stark contrast with the joint Graphical Lasso, which requires a dense coupling among the elements of the precision matrices through the non-decomposable logdet function. Later, we will show how each subproblem can be solved efficiently for different choices of q.

### Example Statistical Guarantees

We examined the statistical properties of Elem-q for SV-GMRFs with two widely-used spatial structures, namely smoothly-changing and sparsely-changing GMRFs. To this goal, we first made two assumptions on the true precision matrices.

Assumption 1 (Bounded norm). There exist constant numbers k₁<∞, k₂>0, and k₃<∞ such that

$${‖ {Θ}_{k}^{⋆} ‖}_{∞} \leq {k}_{1} , {inf}_{w : {‖ w ‖}_{∞} = 1} {‖ {Σ}_{k}^{⋆} w ‖}_{∞} \geq {k}_{2} , {‖ {Σ}_{k}^{⋆} ‖}_{max} \leq {k}_{3}$$

for every k=1, . . . , K.

Assumption 1 is fairly mild and implies that the true covariance matrices and their inverses have bounded norms.

Assumption 2 (Weak sparsity). Each covariance matrix Z, satisfies maxᵢ Eⱼ₌₁^{d}|[Eₖ^{*}]ᵢⱼ|^{P}≤s(p), for some functions: [0,1)→ [symbol] and scalar 0≤p<1.

Informally, we set forth that “the true covariance matrices are weakly sparse” if are s(p)-weakly sparse with s(p)<<d for some 0≤p<1. The notion of weak sparsity extends the classical notion of sparsity to dense matrices. Indeed, except for a few special cases, a sparse matrix does not have a sparse inverse. Consequently, a sparse precision matrix may not lead to a sparse covariance matrix. However, a large class of sparse precision matrices have weakly sparse inverses. For instance, if Θₖ^{*} has a banded structure with small bandwidth, then it is known that the elements of Σₖ^{*}=Θₖ^{*-1} enjoy exponential decay away from the main diagonal elements. Under such circumstances, simple calculation implies that

$$s ( p ) \leq \frac{c}{1 - {p}^{p}}$$

for some constants C>0 and p<1. More generally, a similar statement holds for a class of inverse covariance matrices whose support graphs have large average path length; a large class of inverse covariance matrices with row- and column-sparse structures satisfy this condition. As will be shown later, the weak sparsity parameter s(p) directly controls the sample complexity of our proposed estimator, in this example.

Next, we introduced some notations that simplified our subsequent analysis. Let π:{1, 2, . . . , K}²→{1, 2, . . . , K(K+1)/2} be a fixed, predefined labeling function that assigns a label to each pair (k, l) with l≥k. Let G be a diaconal matrix whose k-th diagonal entry is defined as W_{π}_{^{−1}}₍ₖ₎^{1/q}. Moreover, let A∈ [symbol] ^{K(K-1)/2×K} be the adjacency matrix defined as A(π(k,l),k)=1 and A(π(k,l),l)=−1, for every l>k. Finally, define Θᵢⱼ=[Θ_{1;ij} Θ_{2:ij} . . . Θ_{K:ij}]^{T} and Fᵢⱼ^{*}=[[F*(Σ₁)]ᵢⱼ[F*(Σ₂)]ᵢⱼ . . . [F*(Σ_{K})]ᵢⱼ], for every j≥i. It is easy to see that ∥GAΘᵢⱼ∥_{q}^{q}=Σ_{l>k}Wₖₗ|Θ_{k;ij}−Θ_{l;ij}|^{q} for every j>i, and accordingly, Elem-(i,j,q) can be written concisely as

$${\hat{Θ}}_{ij} = arg {min}_{{Θ}_{ij}} {‖ {Θ}_{ij} - {\tilde{F}}_{ij}^{*} ‖}_{2}^{2} + μ {‖ {Θ}_{ij} ‖}_{1} + γ ‖ GA {Θ}_{ij} {‖}_{q}^{q} .$$

Next we provided sharp statistical guarantees for our proposed method when the precision matrices (Θₖ^{*})ₖ₌₁^{K} change smoothly or sparsely across different distributions.

### Example Smoothly-changing GMRF

We started with our main assumption on the smoothness of the precision matrices.

Assumption 3 (Smoothly-changing SV-GMRFs). There exists a constant D≥0 such that Σ_{l≥k}(Θ_{k;ij}^{*}−Θ_{l:ij})²≥D² for every (i,j).

Informally, we set forth that “SV-GMRF is smoothly-changing” if Assumption 3 if satisfied with a small D. For a smoothly-changing SV-GMRF, it is natural to choose q=2 in Elem-q to promote smoothness in the spatial difference of the precision matrices. Our next theorem characterizes the sample complexity of Elem-q with q=2 for smoothly-changing SV-GMRF. Let Θₘᵢₙ=min{|Θ_{k;ij}|:Θ_{k;ij}^{*}≠0}.

Theorem 1 (Smoothly-changing SV-GMRF). Consider a smoothly-changing SV-GMRF with parameter D, and weakly-sparse covariance matrices with parameter s(p) for some 0≥p≥1. Suppose that the number of samples satisfies

$${n}_{k} ≳ L \frac{log d}{{Θ}_{min}^{2}} ,$$

$$where$$

$$L = max \{ {( \frac{s ( p )}{{κ}_{2}} )}^{\frac{2}{1 - p}} {κ}_{3}^{2} , {( \frac{{κ}_{1} {κ}_{3}}{{κ}_{2}} + D )}^{2} \} .$$

Define nₘᵢₙ=minₖ{nₖ}. Moreover, suppose that F*(Σₖ)=[STᵥₖ(Σₖ)]⁻¹ with vₖ [symbol] k₃\sqrt{log d/nₖ}. Then, the solution obtained from Elem-q with q=2 and parameters

$$γ ≍ \frac{1}{K {‖ W ‖}_{max}} \sqrt{\frac{log d}{{n}_{min}}} ,$$

$$μ ≍ D \sqrt{\frac{log d}{{n}_{min}}} ,$$

satisfies the following statements with probability 1−Kd⁻¹⁰:
Sparsistency: The solution is unique and satisfies supp (Θₖ)=supp (Θₖ^{*}) for every k.

### Estimation Error: The Solution Satisfies

$${‖ {\hat{Θ}}_{k} - {Θ}_{k}^{*} ‖}_{max} ≲ ( \frac{{κ}_{1} {κ}_{3}}{{κ}_{2}} + D ) \sqrt{\frac{log d}{{n}_{min}}} ,$$

$$for every k .$$

For smoothly-changing SV-GMRF, the above theorem provides a non-asymptotic guarantee on the estimation error and sparsistency of the estimated precision matrices via Elem-q with q=2, proving that the required number of samples must scale only logarithmically with the dimension d. Moreover, both the estimation error and the required number of samples decrease with a smaller smoothness parameter D; this is expected since a small value of D implies that the adjacent distributions share more information, and hence, the SV-GMRF is easier to estimate.

### Example Sparsely-Changing GMRF

In sparsely-changing SV-GMRFs, the precision matrices are assumed to change sparsely across different distributions; this is formalized in our next assumption.

Assumption 4 (Sparsely-changing SV-GMRFs). There exists a constant D₀≥0 such that Σ_{l≥k}∥Θ_{k;ij}^{*}−Θ_{l;ij}^{*})∥₀≥D₀ for every (i,j).

Similar to the smoothly-changing SV-GMRFs, we set forth that “SV′GMRFs is sparsely-changing” if it satisfies Assumption 4 with a small D₀. For a sparsely-changing SV-GMRFs, it is natural to choose q=1 in Elem-q to promote sparsity in the spatial difference of the precision matrices. To analyze the statistical property of this problem, we first consider (2) with q=1 and rewrite it as:

$$min {‖ {\tilde{F}}_{ij}^{*} - {Θ}_{ij} ‖}_{2}^{2} + μ {‖ B {Θ}_{ij} ‖}_{1} ,$$

$$where$$

$$B = [ \begin{matrix}\frac{γ}{μ} GA \\ I\end{matrix} ] .$$

The above reformulation is a special case of the generalized Lasso problem introduced by Lee et al. To show the model selection consistency of the above formulation, we next introduce the notion of irrepresentability.

For any fixed (i,j), let S_{B}⊂(1, 2, . . . , K(K+1)/2) be the support of BΘᵢⱼ^{*}, i.e., [BΘᵢⱼ^{*}]_{K}≠0 for every k∈S_{B}. Moreover, let S_{B}^{c}={1, 2, . . . , K(K+1)/2}\S_{B}. Evidently, we have |S_{B}|≥D₀+S₀, where D₀ is introduced in Assumption 4 and S₀ is defined as the maximum number of nonzero elements in Θᵢⱼ^{*}, i.e., S₀=max_{i,j} {∥Θᵢⱼ^{*}∥₀}.

Assumption 5 (Irrepresentability condition (IC), see, e.g., Lee et al., Electronic Journal of Statistics, vol. 9, no. 1, pp. 608-642 (2015). We have

$${‖ {B}_{{S}_{B}^{c} :} {B}_{{S}_{B} :}^{†} sign ( {( B {Θ}_{ij}^{*} )}_{{S}_{B :}} ) ‖}_{∞} \leq 1 - α$$

for some 0<a≥1, where B_{S}_{_{B}}_{^{c}}^{t} is the Moore—Penrose pseudo—inverse of a matrix Bₛ_{_{B:}}.

The irrepresentability condition (IC) entails that the rows of B corresponding to the zero elements of BΘᵢⱼ^{*} must be nearly orthogonal to the other rows. Despite the seemingly complicated nature of IC, classical results on Lasso have shown that it is a necessary condition for the exact sparsity recovery, and hence, cannot be relaxed. Later, we show that this condition is satisfied for our problem under a mild condition on the weight matrix W and parameters μ and γ.

Another quantity that plays a central role in our derived bounds is the so-called compatibility constant defined as

∥B_{S}_{_{B}}_{^{c}}:B_{S}_{_{B}}^{t}∥_{∞}+1.

The compatibility constant K_{IC} is closely related to IC. In particular, if ∥Bₛ_{_{B}}_{^{c}}:B_{S}_{_{B:}}^{t}∥_{∞}≥1−α (which is a slightly stronger version of IC), then κ_{IC}≥2−α. Similar to IC, we will later show that κ_{IC} remains bounded under a mild condition on the weight matrix W. Finally, we define ΔΘₘᵢₙ=min_{k,i,j}{|Θ_{k;ij}−Θ_{k:ij}|:Θ_{k:ij}−Θ_{l:ij}^{*}≠0}.

Theorem 2 (Sparsely-changing SV-GMRFs.). Consider a sparsely-changing SV-GMRFs with parameter Dₒ, and weakly-sparse covariance matrices with parameter s(p) for some 0≥p<1. Suppose that the number of samples satisfies

$${n}_{min} ≳ L \frac{log d}{min \{ {Θ}_{min}^{2} , {ΔΘ}_{min}^{2} \}} ,$$

$$where$$

$$L = \{ {( \frac{s ( p )}{{κ}_{2}} )}^{\frac{2}{1 - p}} {κ}_{3}^{2} , {( \frac{{κ}_{IC} {κ}_{1} {κ}_{3}}{{κ}_{2} α} )}^{2} ( {‖ W ‖}_{max} {D}_{0} + {S}_{0} ) \} .$$

Define nₘᵢₙ=minₖ{nₖ}. Suppose that F*(Σₖ)=[STᵥₖ(Σₖ)]⁻¹ with Vₖ [symbol] k₃ \sqrt{log d/nₖ}. Moreover, suppose that the weight matrix W and parameters ii and y are chosen such that IC (Assumption 5) is satisfied. Then, the solution obtained from Elem-q with q=1 and parameter

$$μ ≍ \frac{{κ}_{IC} {κ}_{1} {κ}_{3}}{{κ}_{2} α} \sqrt{\frac{log d}{{n}_{min}}}$$

Satisfies the following statements with probability 1−Kd⁻¹⁰:
Sparsistency. The solution is unique and satisfies supp(Θₖ)=supp(Θ_{κ}^{*}) for every κ and supp(Θ_{κ}−Θₗ)=supp(Θₖ^{*}−Θₗ^{*} for every k>l.
   Estimation error. For every (i,j), the solution satisfies

$${‖ {\hat{Θ}}_{ij} - {Θ}_{ij}^{*} ‖}_{2} ≲ ( \sqrt{{‖ W ‖}_{max} {D}_{0}} + \sqrt{{S}_{0}} ) \frac{{κ}_{IC} {κ}_{1} {κ}_{3}}{{κ}_{2} α} \sqrt{\frac{log d}{{n}_{min}}} .$$

The above theorem characterizes the sample complexity of inferring sparsely-changing SV-GMRFs, showing that the sparsity pattern of the precision matrices and their differences can be recovered exactly, given that the number of samples scale logarithmically with the dimension and the problem satisfies IC. Evidently, our result relies on IC and K_{IC} being small. This leads to a follow-up question: how restrictive are these conditions in practice? Our next proposition shows that both conditions hold if y and are selected such that μ≥γ≥2μ and W_{κI} is the same for every K>l.

Proposition 1. Suppose that 0<μ≥γ≥2μ and W_{κI} is the same for every κ>l. Then, 1≥κ_{IC}≥5 and IC holds with α=μ/γ.

Proposition 1 can be easily extended to general choices of W. In particular, suppose that W=π11^{T}+E for some τ>0, where 1 is the vector of ones. Then, Proposition 1 combined with a simple matrix perturbation bound reveals that

$$α \geq 1 / 2 - 𝒪 ( {‖ E ‖}_{max} ) ,$$

$$and$$

$$1 \leq {κ}_{IC} \leq 5 + 𝒪 ( {‖ E ‖}_{max} ) .$$

In other words, IC holds and κ_{IC} remains bounded, provided that ∥E∥ₘₐₓ= [symbol] (1), that is, the elements of the weight matrix W do not vary too much. Later in our numerical experiments, we will show that such choices of W provide the best statistical results on both synthetically generated as well as gene expression datasets.

### Example Parameter Tuning and Implementation

W we explain different implemental aspects of this example implementation. First, we focused on parameter tuning.

Parameter tuning: To obtain a solution for Elem-q, we first need to fine-tune the parameters μ, γ, νₖ, W based on the available data samples. Recall that, for every pair (k, l), the value of Wₖₗ⁻¹ can be interpreted as the “distance” between precision matrices for distributions k and l. Intuitively, Θ_{κ}^{*} and Θₗ^{*} are close if their corresponding covariance matrices z; and E; are close. For synthetically generated data, we have observed that the distance between any pair of sample covariance matrices Dₖₗ=∥Σₖ−Σ_{f}∥_{f2} can be directly used to provide reasonable values for the weight matrix W. In particular, we have observed throughout our experiments that the choice of Wₖₗ=1/(1+Dₖₗ) for every k≠l leads to a desirable performance for our estimator. Indeed, this choice of W remains well-conditioned in all of our experiments, which is aligned with our discussion after Proposition 1. It is worth noting that in the high-dimensional setting, Dₖₗ may not be a reliable estimate of the distance between the true covariance matrices Σₖ^{*} and Σₗ^{*}. Nonetheless, our numerical results reveal that the proposed estimation framework is robust against possible inaccuracies in Dₖₗ.

Next, we explain our approach for fine-tuning the parameters μ, γ, and νₖ. The parameter controls the sparsity of the estimated precision matrices, whereas y penalizes their differences. Moreover, νₖ is the threshold used in the proposed approximate backward mapping. In Theorems 1 and 2, we provide an explicit value for these parameters that depend on the parameters of the true solution, which are not known a priori. Without any prior knowledge on the true solution, these parameters can be selected by minimizing the extended Bayesian Information Criterion (BIG):

$$( \hat{u} , \hat{γ} , \hat{v} ) = arg {min}_{u , γ , v} BIC ( u , γ , v ) ,$$

$$where$$

$$\begin{matrix}BIC ( u , γ , v ) := {\sum }_{k = 1}^{K} {n}_{k} [ Tr ( {\hat{\sum }}_{k} {\hat{Θ}}_{k} ( u , γ , v ) ) - log det {\hat{Θ}}_{k} ( u , γ , v ) ] \\ + log ( {n}_{k} ) {df}^{( k )} + 4 {df}^{( k )} log d ,\end{matrix}$$

In the above definition, Θₖ(μ,γ,ν) is the optimal solution of (Elem-q) with parameters (μ,γ,ν). Moreover, df^{(k)} is defined as the number of nonzero elements in Θₖ(μ,γ,ν). Theorems 2 and 1 suggest that γ=C₁\sqrt{log d/nₘᵢₙ}, μ=C₂\sqrt{log d/nₘᵢₙ}, and νₖ=C₃\sqrt{long d/nₖ}, where C₁, C₂, and C₃ are constants that depend on the true solution. To pick the parameters (μ,γ,ν). An important benefit of our method compared to other MLE-based approaches is the efficient solvability of (Elem-q) for each choice of parameters, which in turn makes the proposed grid search feasible in practice. The details of our algorithm are presented next.

Algorithm: Next, we explain a general algorithm for solving Elem-q. As mentioned before, Elem-q decomposes over different coordinates (i, j), where each subproblem can be written as Elem-(i, j, q). This decomposition leads to a parallelizable algorithm, where each thread solves Elem-(i, j, q), for a subset of the coordinates (i, j). This approach is outlined in Algorithm 1.

Algorithm 1: General algorithm for solving Elem-q

1 Data: Data samples {xᵢ^{k}}, parameters (μ, γ, νₖ), and weight matrix W.
2 Result: Solution {\hat{Θ}ₖ}ₖ₌₁^{K} for Elem-q.
3 Compute the sample covariance matrix \hat{Σ}ₖ for every k = 1, ... , K.
4 Compute the approximate backward mapping
\tilde{F}*(\hat{Σ}ₖ) = [ST_{ν}_{ₖ}(\hat{Σ}ₖ)]⁻¹ for every k = 1, ... , K.
5 for every (i, j) do
6 Obtain \hat{Θ}ᵢⱼ by solving Elem-(i, j, q).
7 end

Next, we analyze the computational cost of each step of our proposed algorithm. Given n_{κ} number of samples, the sample covariance matrix Σ_{K} can be computed in [symbol] (n_{κ}d²) time and memory (Line 3). Moreover, given each sample covariance matrix, the approximate backward mapping can be obtained by an element-wise soft-thresholding followed by a matrix inversion, which can be done in [symbol] (d³) time and memory (Line 4). Finally, for each (i,j) and the choices of q=1, 2, the subproblem Elem-(i,j,q) can be reformulated as a linearly constrained convex quadratic problem. Suppose that W has nnz number of nonzero elements. Then, each subproblem can be solved in [symbol] (nnz³).

Moreover, assuming that the algorithm is parallelized over M machines, the total complexity of solving all subproblems is [symbol] (

$$( \frac{{d}^{2}}{M} )$$

nnz³). Below we show that our proposed processes are extremely efficient in practice.

### EXAMPLES

We evaluated the performance of various examples of the techniques herein on a synthetically generated dataset, as well as a Glioblastoma spatial transcriptomics dataset.

### Synthetically Generated Dataset

We used a synthetically generated dataset to compare the statistical performance of an example implement of the current techniques with two conventional estimators: a fused graphical lasso (FGL) and FASJEM. The FGL approach is an MLE-based approach augmented by a regularizer to promote spatial similarity among different distributions. On the other hand, the FASJEM approach uses an elementary estimator framework, but varies greatly from the present techniques by using a quite different regularization term. By comparing the estimated parameters with their true counterparts, we were able to show that, in this example, the present techniques greatly outperformed both FGL and FASJEM in recovering the true precision matrices.

Data generation was designed to imitate gene expression profiles from a synthetically generated co-expression network. The goal was to generate the data synthetically from a known distribution, and then evaluate the performance of the estimated parameters by comparing them to the ground truth.

We simulated the true precision matrices for a distinct clusters (populations) with varying level of similarity. Within each cluster, we assumed that the graph representing the true precision matrix has a disjoint modular structure, with power law degree distribution for nodes within each module. Specifically, we split d genes into M modules, with d/M genes per module generated based on Barabasi-Albert model. Within each cluster, the modules were simulated independently and concatenated to produce a block diagonal matrix, which is treated as the true precision matrix for the corresponding cluster.

In order to simulate population-specific precision matrices, we first generated a random spanning tree over clusters. Starting with the root population, we generated M modules, and in each module, we randomly generate a graph with d/M vertices according to the Barabasi-Albert model. Based on the adjacency matrix of this graph, we selected the edge weights uniformly from [−1,−0.4]U[0.4,1]. To ensure the positive semi-definiteness of the constructed precision matrix, we used 1.1 times the sum of the absolute values of all off-diagonal elements in each row as the value of the diagonal elements in that row. The simulated modules were used to generate the population-specific block-diagonal precision matrix. We then traversed the spanning tree from the root cluster and, at every new cluster, construct the precision matrix by perturbing its parent cluster.

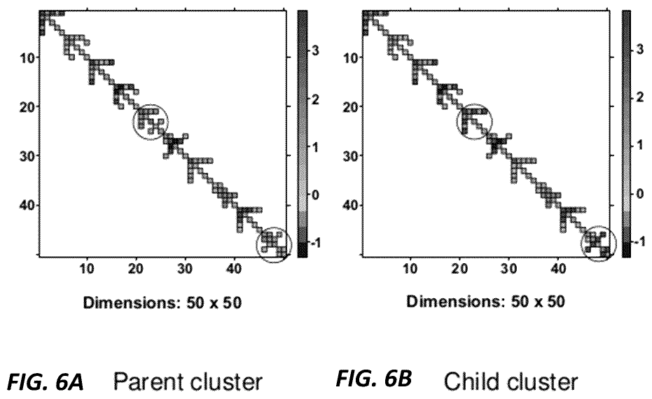

We considered two types of perturbations: (i) edge weight perturbation; and (ii) edge reconnection. To perform Type (i) perturbation, we sampled a subset of the M modules at the parent cluster and added a uniform perturbation from the interval [−0.04,0.04] to the non-zero edges. For Type (ii) perturbation, we replaced one of the M modules with a newly simulated one following a power-law degree distribution. Thus, at every cluster, the precision matrix is slightly perturbed relative to its parent, and the precision matrix differences accumulate, which means that the number of different edges of two precision matrices increases with their distance. FIGS. 6A and 6B illustrate the precision matrices for the two adjacent clusters, with a parent cluster in FIG. 6A and a child cluster in FIG. 6B. Having simulated the precision matrices, at every cluster κ, we next collected n_{κ} samples from a zero-mean Gaussian distribution with the constructed precision matrix.

### Example Experiment 1: Varying Number of Samples

In a first experiment, we examined techniques by varying the number of samples. In this experiment, we demonstrated that the present techniques perform well over an order of magnitude variation in number of samples researchers may have per subpopulation. Such situations can offer because clinicians are usually dealing with a very high-dimensional setting, where the number of variables is much larger than sample size.

In the example, we fixed K=5, d−250, and M=5, and compared the performance of Equation Elem-q above with FGL and FASJEM with varying number of samples n_{κ}. We compared the estimation accuracy in terms of Recall=TP/(TP+FN), Precision=TP/(TP+FP), and F1-score=2 (Recall×Precision)/(Recall+Precision), where TP, FN, and FP correspond to the number of correctly identified nonzero elements (true positive), incorrectly identified zero elements (false negative), and incorrectly identified nonzero elements (false positive), respectively. To fine-tune the weight matrix w and the parameters (μ,γ,ν_{κ}), we used the distance measure and BIC approach described in reference to parameter tuning and implementation. Moreover, we used the same BIC approach to fine-tune the parameters of FGL and FASJEM, for comparison's sake.

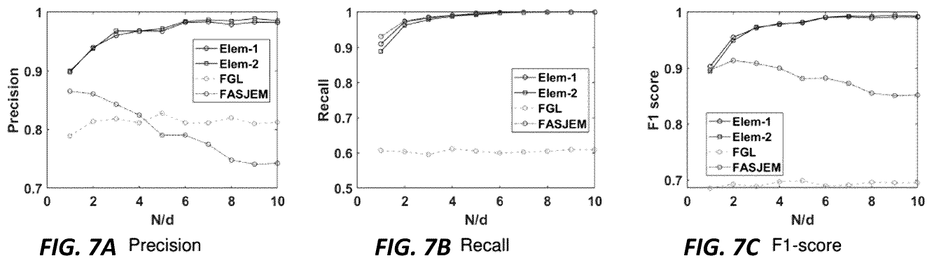

FIGS. 7A-7C illustrate plots of the performance of different estimation methods. It can be seen that Elem-q with q=1, 2 (denoted as Elem-1 and Elenn-1) perform almost the same, and they both outperform FASJEM and FGL in terms of the Precision (FIG. 7A) and F1 scores (FIG. 7C). On the other hand, the Recall score (FIG. 7B) for FASJEM is artificially high due to the underestimation of the regularization parameters via BIC, which in turn leads to overly dense estimation of the precision matrices.

### Example Experiment 2: Varying Dimensions

In another experiment, we analyzed the performance of an example of the present techniques for different dimensions d. In this experiment, we demonstrated accurate performance of the present techniques in high-dimensional settings, with millions of parameters to be estimated. This arises, for example, when clinicians want to estimate a gene-network at the transcriptome-scale with thousands of genes.

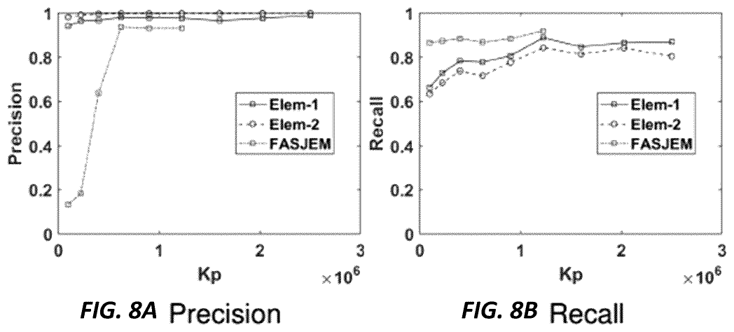

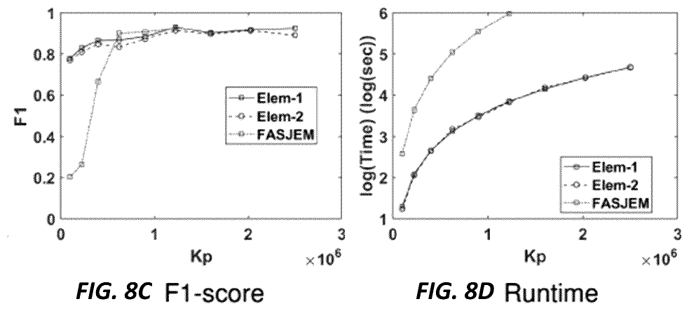

In this example, we considered a high-dimensional regime where d is significantly larger than the number of available samples nₖ. We fix K=5 and set nₖ−d/2. The parameters μ,γ,ν_{κ} and the weight matrix W were tuned as above. FIG. 8A-8D depict plots of the Precision, Recall, and F1-score, as well as the runtime, respectively, of the proposed techniques and FASJEM with respect to Kp=Kd(d+1)/2 which ranges from 10⁵ to 2.5×10⁶. For these instances, FGL did not converge within 10 minutes even for the smallest instance with d=200. Therefore, FGL was omitted from our subsequent experiments.

The runtime of the proposed techniques scales almost linearly with p, with the largest instance solved in less than 2 minutes. On the contrary, FASJEM has an undesirable dependency on p, with a runtime exceeding 10 minutes for medium-scale instances of the problem. The linear time of the present techniques with respect to p is believed to be due to its decomposable nature of over different coordinates of the precision matrices.

### Example Experiment 3: Varying Number of Clusters

As discussed herein, the number of clusters correspond to the number of precision matrices (or the number of subgroups) that are to be inferred. For example, in spatial-genomics, this may correspond to the number of locally homogeneous regions (like niches) within each tissue, which could differ depending on the heterogeneity within the tissue microenvironment. Even more broadly, in the context of structured-omics data, this may could represent localized subregions (like a subgraph of a graph network, a spatial subregion or a window/range of time within a larger time series).

In another experiment, we examined the performance of an example of the present techniques by varying the number of clusters K. In this experiment, we examined example performance of the present techniques where there are a large number of related subpopulations whose networks need to be jointly estimated. This allows us flexibility in increasing the resolution of clustering of the original dataset, if we want to look at really fine changes in biology.

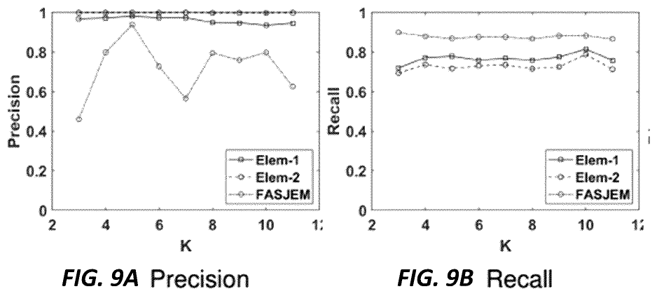

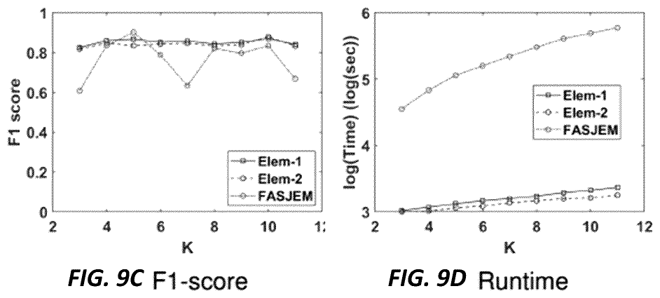

In this example, we fixed d=500, M=10 and n_{κ}=250, and used the same tuned parameters in Experiment 2. FIGS. 9A-9D illustrate plot showing the Precision, Recall, and F1 score for the proposed techniques and FASJEM, as well as their runtime with respect to K. Similar to the previous experiments, both Elem-1 and Elem-2 outperform FASJEM in terms of the estimation accuracy. Moreover, it can be seen that in practice, the runtime of Elem-1 and Elem-2 scale almost linearly with K.

### Application to Glioblastoma Spatial Transcriptomics

We applied the present techniques to generate regulatory networks on glioblastoma spatial transcriptomic data. In this example, we assayed spatial gene expression profiles using the Visium platform from a primary patient tumor area showing high perfusion signal in diffusion MRI. The Visium slides profile gene expression from a 6.5×6.5 mm section of tumor tissue. We sampled two adjacent tissue sections from this region, yielding 6158 spots with expression data for the sample.

We integrated data from adjacent tissue slices using the reciprocal PCA method in Seurat (see, e.g., Hao et al., Cell, vol. 184, no. 13, pp. 3573-3587 (2021)). We used the dimension reduction algorithm PHATE (see, e.g., Moon et al., Nature Biotechnology, vol. 37, no. 12, pp. 1482-1492 (2019)) to obtain a 3D embedding of the integrated counts data that captures the expression similarity of spots. To cluster the data in a spatially informed manner, we computed two separate distance matrices between spots, from their PHATE embedding and tissue coordinates. We perform upper quantile normalization of the distance matrices based on their 75th quantile to ensure that both expression and spatial distances are in the same scale, and use their sum to define pairwise distances between spots.

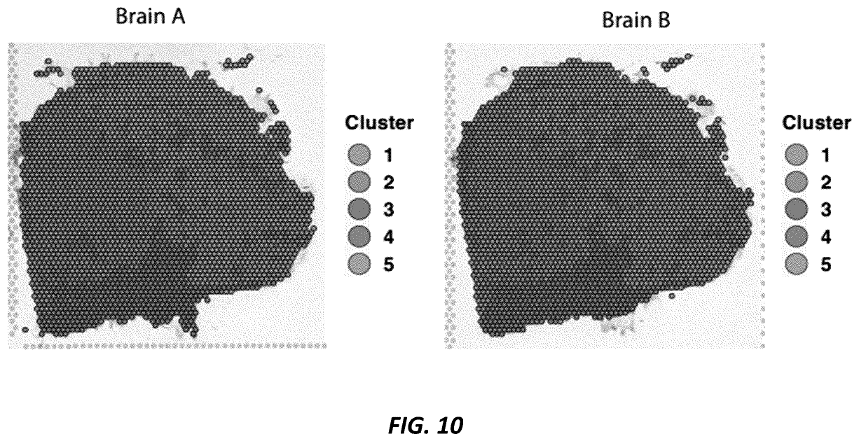

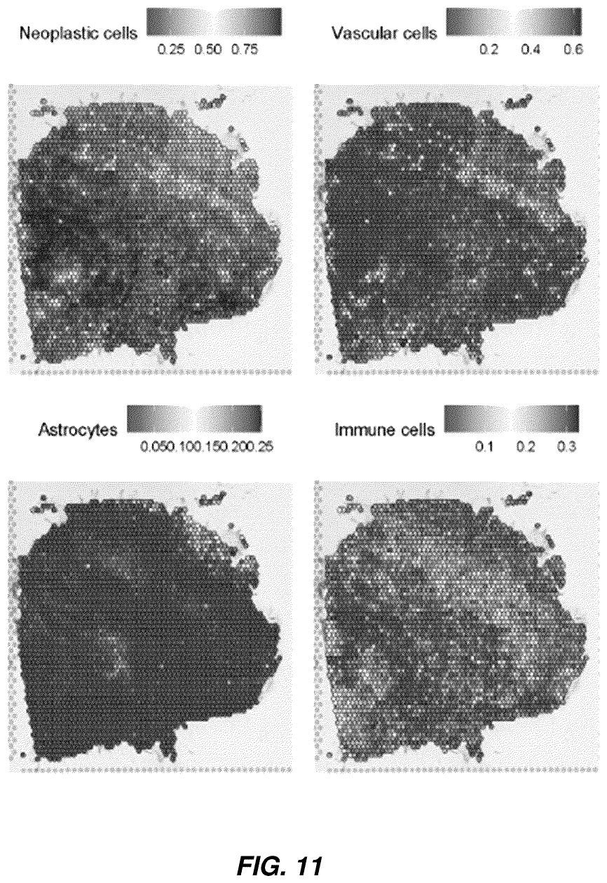

This dissimilarity matrix was used as input for PAM clustering. The optimal number of clusters (k=5) was identified using the Calinski-Harabasz criterion (see, e.g., Calinski et al., “Communications in Statistics-theory and Methods, vol. 3, no. 1, pp. 1-27 (1974)), with the resulting clusters shown in FIG. 10. To understand biological characteristics of these clusters, and to aid in downstream interpretation of inferred networks, we performed spot deconvolution using the RCTD algorithm (see, e.g., Cable et al., Nature Biotechnology, pp. 1-10 (2021)). Since spots in the Visium microarray have a resolution of about 60 μm, they could be composed of multiple cell types. Therefore, we used annotated single cell RNASeq dataset to identify compositional differences between the regions. FIG. 11 shows the deconvolution results. As shown, the sample tissue is primarily composed of neoplastic cells. Referring back to FIG. 10, cluster 4 represents a distinctive pen-vascular niche with significant immune infiltration, and cluster 5 has a high proportion non-neoplastic cells. The clusters 1, 2, and 3 are sub-clusters of tumor cells from different microenvironments in the tissue. They lie at different distances from vasculature, showing varying degrees of adaptation of hypoxia.

With the characteristics of the clusters established, we sought to understand how the gene interaction network varied in different regional microenvironments of this tumor section.

### Data Preparation for Regulatory Network Inference

We identified the top 2500 genes showing significant spatial trends in their expression determined using the SparkX algorithm (see, e.g., Sun et al., Genome Biology, vol. 22, no. 1, pp. 1-25 (2021)). Since gene expression counts data followed a negative-binomial distribution rather than Gaussian, we used the non-paranormal transformation to make the data amenable for analysis using a Gaussian graphical modeling framework. This procedure worked by non-parametrically estimating monotone functions fj such that the transformed variables f(X)=(f₁(X_{I}), f₂(X₂), . . . fₚ(Xₚ)) are normally distributed (f(X)˜N(μ,Σ)). Importantly, the transformation preserved the independence relations between the variables Xⱼ, so the graph structure is not altered. We use the R package huge for transforming the normalized spot level counts data.

Inter-cluster similarity constraints for network inference were imposed based on pairwise distances between cluster medoids. Since our clustering was based on proximity in both expression and spatial coordinates, we believe this is a reasonable biological constraint to impose on the network inference algorithm. We transformed distances into similarity using the formula

$${W}_{i , j} = \frac{1}{1 + {d}_{i , j}} ,$$

where d_{1,1} is the sum of expression and spatial distance between medoids for clusters i, j.

We used BIG criterion to identify the optimal sparsity and similarity constraint parameters for the network inference. Network inference showed that of the 2500 genes considered, 1180 had an edge in at least one cluster.

### Regulatory Network Structures and Biological Significance

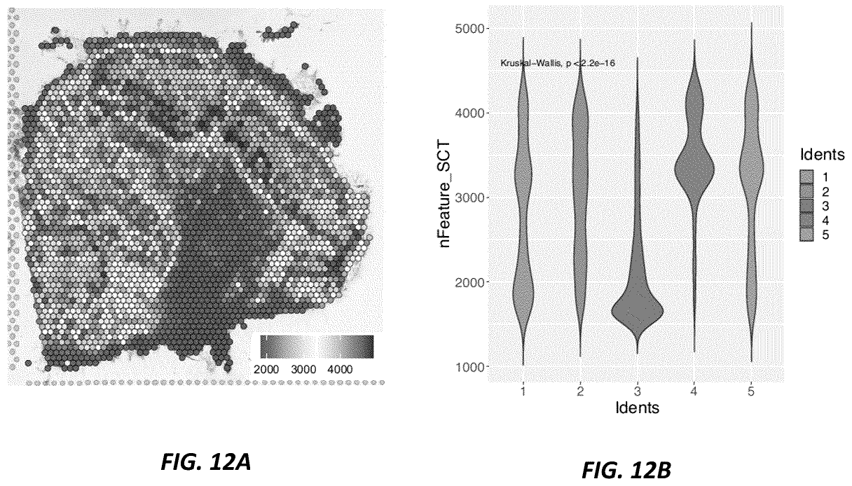

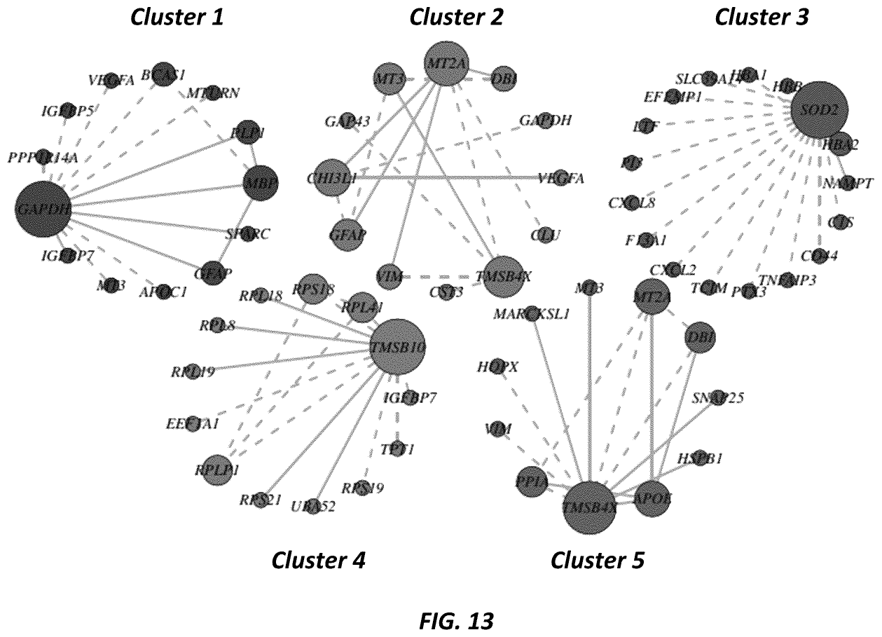

The tissue section showed large variations in number of expressed genes between clusters (visualized in FIGS. 12A and 12B), reflecting biological trends in transcriptional activity across the tumor slide. The number of inferred edges per cluster were respectively 4511, 13785, 446, 8400 and 4534. The spatial region in Cluster 3 has significantly fewer detected genes than other regions explaining the very low connectivity in its network. To compare the extent of similarity between the inferred networks, we used the DeltaCon algorithm (see, e.g., Koutra et al., Proceedings of the 2013 SIAM International Conference on Data Mining, SIAM, pp. 162-170 (2013)), a statistically principled and scalable Intergraph similarity function. We saw that inferred networks from the different clusters share little similarity, with Clusters 1 and 3 having the maximum pairwise similarity of 0.27.

The network in Cluster 4 was maximally different from the other regions. This is as expected, given that this region is compositionally most unique.

FIG. 13 illustrates five inferred gene regulatory networks generated by the biological regulatory network platform (more specifically by a report generator thereof) showing the top 15 strongest edges in each cluster, with node sizes scaled by their respective degree and negative interactions shown as dotted lines. We can immediately see that each cluster has distinct underlying regulatory interactions driving their transcriptional states, even if they appear compositionally homogeneous.

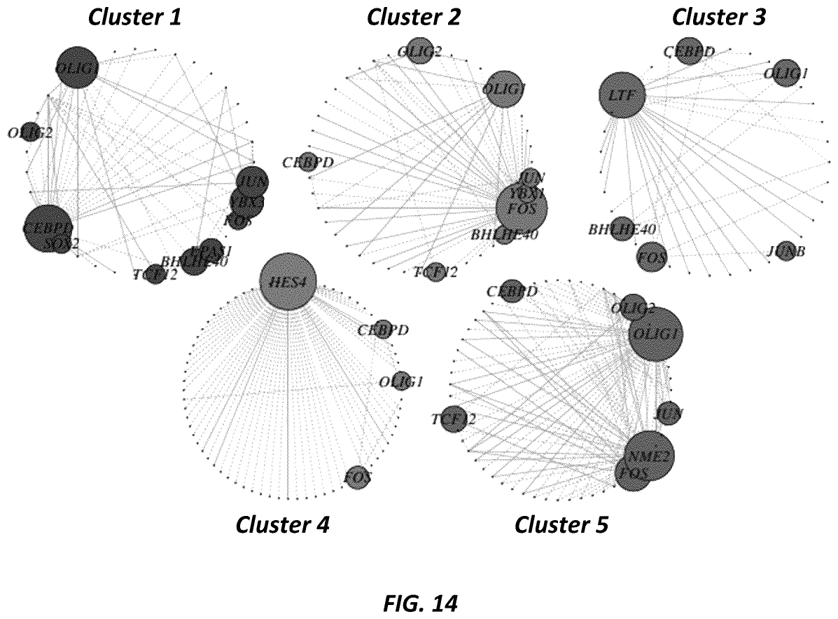

Transcription factors (TFs) are proteins that play a dominant role in regulating gene expression networks of cells and are particularly important in driving tumor growth and evolution. By binding to regulatory regions of target genes, transcription factors are responsible for regulating gene expression and thereby controlling cell states. Regulatory interactions involving TFs are therefore of particular interest in understanding gene networks. We highlight these interactions in FIG. 14. Since Cluster 2 has an order of magnitude more edges than other networks, we highlight only the top 100 edges. Comparing FIGS. 13 and 14, FIG. 13 only shows the top 25 strongest edges for each network. Each of these networks have thousands of edges between the genes. In FIG. 14, we subset the inferred networks to only include edges to and from TFs and highlight the strongest interactions involving TFs. In this way, FIG. 14 is essentially zooming in on regulatory edges involving TFs, as these are the major drivers of the biology.

Reviewing FIG. 14, frequently highlighted TFs active across different regions include the AP1 family TFs FOS and JUN, which are known downstream effectors of the Mesenchymal state in Gliomas, and other master regulators such as CEBPD and oligodendroglial lineage factors OLIG1 and OLIG2. We also see significant activity of SOX2 in Cluster 1, a known driver of sternness features and radiation-resistance in Gliomas. Cluster 3 shows significant activity of Lactotransferrin (LTF), which encodes an iron-binding protein with known innate immune and tumor-suppressive activity. Interestingly, this gene has also been characterized as being an upstream master regulator of different GB subtypes, warranting further exploration of this gene in driving tumorigenesis in GB.

The TF network in Cluster 4, which represents the perivascular niche, is most different from the other regions, as expected given its unique microenvironment. This region shows prominent activity of HES4, a known downstream effector of the NOTCH signaling pathway that is known to inhibit cell differentiation and helps maintain the sternness features in Gliomas. HES4 specifically regulates proliferative properties of neural stem cells, and reduces their differentiation. This is a very promising observation, given that the perivascular niche is known to harbor therapy-resistant glioma stem cells whose properties are critically driven through NOTCH signaling. Cluster 5 has dominant activity of NME2, a nucleotide diphosphate kinase enzyme involved in cellular nucleotide metabolism and DNA repair. The NME2 protein has also been identified to be a highly specific Tumor-associated antigen in IDH mutant Gliomas. By studying the regulatory networks in each cluster, we are thus able to infer differential activity of different master regulators in distinct micro-environmental niches across the tissue section.

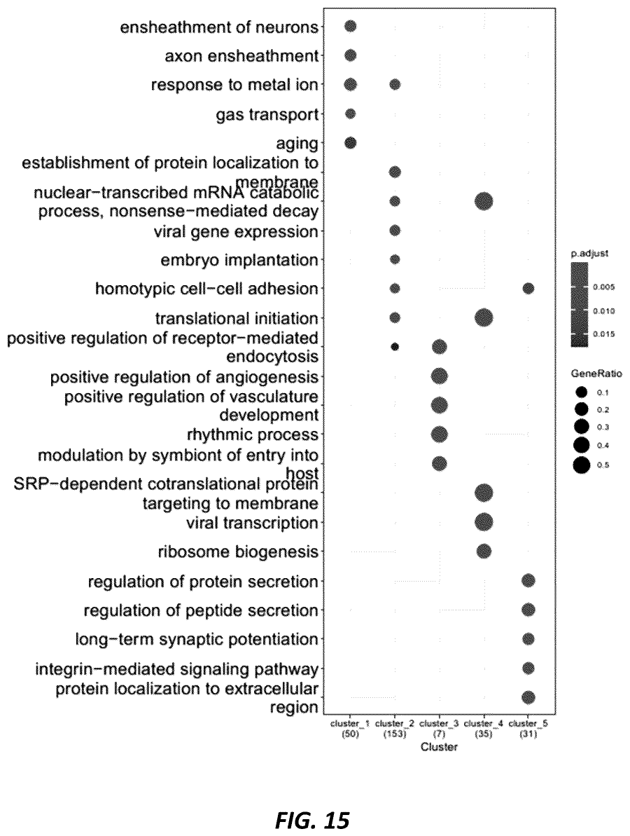

To aid with biological interpretation, the regulatory network informed treatment prediction module 150 of FIG. 2 an/or the biological network informed treatment analyzer 326 of FIG. 4 were configured to compute different centrality measures (degree, betweenness, closeness, Eigen and PageRank centrality) for genes in each network, each of which measures a different aspect of importance of nodes. In an example, the analyzer 326 reduced each network to its set of unique edges, and considered the top 10% of nodes by each centrality measure to be hub genes. From there, the analyzer 326 generated a gene ontology enrichment analysis resulting in a report with the cluster-specific hub genes shown in FIG. 15. This highlights specialized activity of different biological processes in each cluster. Cluster 1 shows an enrichment for neuronal differentiation related genes. Cluster 2 specific hub genes are associated with metabolic and biosynthetic processes. Cluster 3 hubs are associated with innate immune responses, in agreement with our observation that LTF is a major TF in this network. Cluster 4 has a large number of ribosomal genes with high connectivity. High levels of ribosomal protein activity has been shown to be associated with promoting sternness characteristics of Gliomas, and we see it to be a defining characteristic of the pen-vascular niche. Cluster 5 shows enrichment for neuronal processes like synaptic transmission, consistent with the presence of significant astrocytic population in this region.

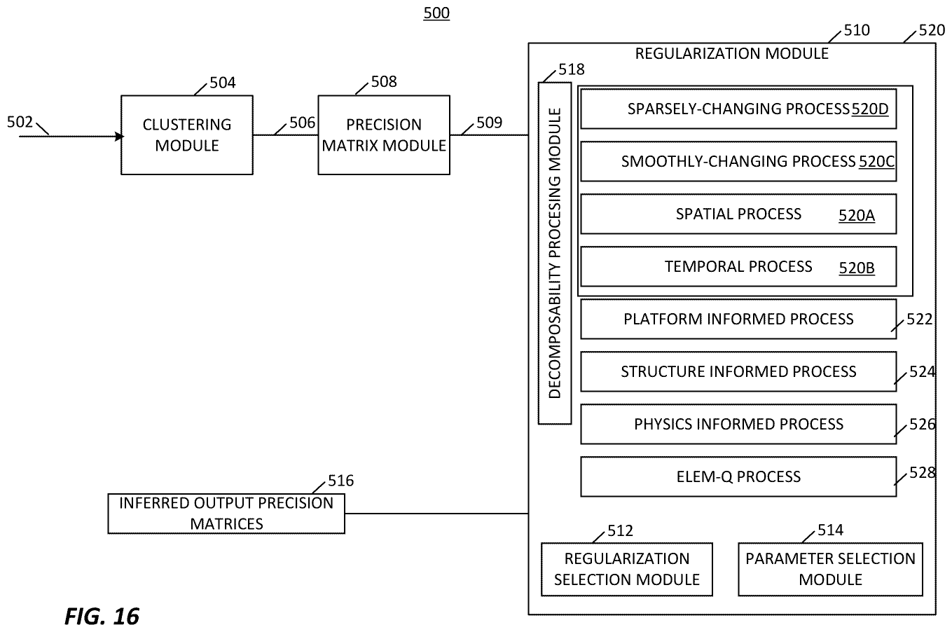

As noted above, the techniques herein may be applied to infer regulatory networks from any suitable structured-omic data using inference and optimization processes built upon tunable regularization stages. While FIG. 4 illustrates an example with two tunable regularization processes, FIG. 16 illustrates another example architecture 500 for implementing the techniques herein. In the illustrated example structured-omic data 502, such as is provided to a clustering module 504 that performs a clustering process on the structured-omic data generating plurality of candidate clusters 506. Each candidate cluster 506 corresponds to a different regulatory network of a subpopulation in the structured-omic data 502. Each candidate cluster 506 may a plurality of nodes and edges between nodes, each node corresponding to a regulator and each edge corresponding to an interaction between regulators connected by each edge. Each candidate cluster 506 is converted to a candidate precision matrix, for example, in a precision matrix module 508, that sends the candidate precision matrixes 509 to a regularization stage 510.

In the illustrated example, the regularization stage 510 contains a plurality of different types of regularization processes, each configured to compare different types of candidate precision matrices using embedded optimizations to infers one or more output precision matrices each output precision matrix corresponding to different subpopulations in the structured-omic data 502. The regularization stage 510 is tunable to one or more of these regularization processes defining the optimization, through a regularization selection module 512. Further, the regularization stage 510 is tunable, through a parameter selection module 514, to select a weighting factor imposing a similarity between candidate precise matrices that is applied during the optimization. The regularization module 510 generates one or more output precision matrices 516 that may be converted to one or more output inferred regulatory networks, using a report generator as described above, or through configuring the regularization module 510 to perform such conversions.

In the illustrated example, the regularization module 510 includes a decomposability processor module 518 at a front end. That module 518 performs a decomposability process on the received candidate precision matrices 509. For example, the decomposability module 518 may decompose the problem into smaller subproblems, each defined over different coordinates of the precision matrix. This enables each subproblem to be solved faster and more efficiently.

In the illustrated example the regularization module 510 includes one or more of each of a domain informed regularization process 520, a platform informed regularization process 522, a structure informed regularization process 524, and a physics informed regularization process 526. In the illustrated example, the domain informed regularization process 520 actually contains a plurality of processes, including a spatial regularization process 520A and a temporal regularization process 520B. Further, the domain informed regularization process 520 contains a smoothly-changing regularization process 520C and a sparsely-changing regularization process 520D, similar to the example architecture shown in FIG. 4. In some examples, the parameter tuning module 514 is used to select one of the tuning variables for such processes, such as by selecting A, y, and μ values.

The regularization stage 510 further includes a regularization process 528 that contains a backward mapping deviation as an I2 regularization, an absolute regularization as an I1 regularization, and a tunable spatial regularization. In the illustrated example, the process 528 applies the expression Elem-q in performing precision matrix optimizations and inferences. In some such examples, the tuning of modules 512/514 may include determining a q tuning factor having values corresponding to the one or more regularization processes and determining a weighting factor for imposing a spatial similarity between candidate precision matrices.

It will be appreciated that any of the regularization processes shown may be implemented as a trained machine learning model.

The following list of aspects reflects a variety of the embodiments explicitly contemplated by the present disclosure. Those of ordinary skill in the art will readily appreciate that the aspects below are neither limiting of the embodiments disclosed herein, nor exhaustive of all of the embodiments conceivable from the disclosure above, but are instead meant to be exemplary in nature.

Aspect 1. A method of inferring a regulatory network of interactions of regulators in structured-omic data, the method comprising: obtaining, at one or more processors, the structured-omic data for a sample, the structured-omic data comprising expression data and corresponding location data; performing, at the one or more processors, a clustering process on the structured-omic data to generate a plurality of candidate clusters, each candidate cluster corresponding to a different regulatory network of a subpopulation in the structured-omic data, each candidate cluster comprising a plurality of nodes and edges between nodes, each node corresponding to a regulator and each edge corresponding to an interaction between regulators connected by each edge; for each candidate cluster, generating a precision matrix, at the one or more processors, establishing a plurality of candidate precision matrices; providing, at the one or more processors, the plurality of candidate precision matrices to a regularization stage, and in the regularization stage comparing the candidate precision matrices using an optimization that infers one or more output precision matrices each output precision matrix corresponding to different subpopulations in the structured-omic data, wherein the regularization stage is tunable to one or more regularization processes defining the optimization and tunable to select a weighting factor imposing a similarity between candidate precise matrices that is applied during the optimization; and converting, at the one or more processors, the one or more output precision matrices to one or more output inferred regulatory networks.

Aspect 2. The method of Aspect 1, wherein the regulator of each node of the candidate clusters is a biological regulator selected from the group consisting of DNA, RNA, a gene, a protein, proteome, a metabolite, or any combination thereof.

Aspect 3. The method of Aspect 1, wherein the regulator of each node of the candidate clusters is a biological regulator of selected from the group consisting of genomic data, epigenomic data, chromatin state data, transcriptomic data, proteomic data, metabolomics data, spatial multiplexed imaging data or any combination thereof.

Aspect 4. The method of Aspect 1, wherein structured-omic data comprises RNASeq data, spatially encoded RNASeq data, mRNA data, DNA data, miRNA, epigenomic data, proteomic data, spatial transcriptomic-epigenomic data, integrated transcriptomic-proteomic data, integrated multiplexed IF-transcriptomic data, or spatial Clustered Regularly Interspaced Short Palindromic Repeats (CRISPR)/CRISPR Associated (CAS) screens.

Aspect 5. The method of Aspect 1, wherein each of the candidate clusters is a gene regulatory network and wherein each of the one or more output inferred regulatory networks is a gene regulatory network.

Aspect 6. The method of Aspect 1, wherein each of the one or more output inferred regulatory networks corresponds to a spatially different subpopulation in the sample, a temporally different subpopulation in the sample, or a spatially-temporally different subpopulation in the sample.

Aspect 7. The method of Aspect 1, wherein the sample comprises tissue taken from different organs within a subject.

Aspect 8. The method of Aspect 1, wherein the sample comprises cancer tissue.

Aspect 9. The method of Aspect 8, wherein the sample comprises primary cancer tissue and secondary cancer tissue.

Aspect 10. The method of Aspect 1, wherein the one or more output inferred regulatory networks differ in a molecular function, a biological process, or cellular components.

Aspect 11. The method of Aspect 1, wherein the interaction between regulators is an activation interaction or a suppression interaction between nodes defining the edge.

Aspect 12. The method of Aspect 1, wherein the regularization stage comprises a plurality of regularization processes.

Aspect 13. The method of Aspect 1, wherein the one or more regularization processes comprise a domain-informed regularization process, knowledge-informed regularization process, a platform-informed regularization process, structure-informed regularization process, a physics-informed regularization process.

Aspect 14. The method of Aspect 1, wherein the regularization stage comprises a spatial regularization process or a temporal regularization process.

Aspect 15. The method of Aspect 1, wherein the regularization stage comprises a smoothly-changing regularization process and a sparsely-changing regularization process. Aspect 2. The computer-implemented method of Aspect 1,

Aspect 16. The method of Aspect 15, wherein the smoothly-changing regularization process is an I2 regularization process that is tunable by selecting λ, γ, and μvalues.

Aspect 17. The method of Aspect 15, wherein the sparsely-changing regularization process is an I1 regularization process that is tunable by selecting λ, γ, and μvalues.

Aspect 18. The method of Aspect 1, wherein the regularization stage comprises a backward mapping deviation as an I2 regularization, an absolute regularization as an I1 regularization, and a tunable spatial regularization.

Aspect 19. The method of Aspect 1, wherein the regularization stage performs a decomposability process prior to the optimization.

Aspect 20. The method of Aspect 1, wherein the regularization stage is tunable through (i) a q tuning factor having values corresponding to the one or more regularization processes and (ii) a weighting factor for imposing a spatial similarity between candidate precision matrices.

Aspect 21. The method of Aspect 1, wherein the regularization stage applies the optimization using the expression:

$$\begin{matrix}{\{ {\hat{Θ}}_{k} \}}_{k = 1}^{K} = arg {min}_{{\{ {\hat{Θ}}_{k} \}}_{k = 1}^{K}} {\underbrace{{\sum }_{k = 1}^{K} {‖ {Θ}_{k} - {\tilde{F}}^{*} ( {\hat{\sum }}_{k} ) ‖}_{{ℓ}_{2}}^{2}}}_{backward mapping deviation} \\ + {\underbrace{μ {\sum }_{k = 1}^{K} {‖ {Θ}_{k} ‖}_{{ℓ}_{1}}}}_{absolute regularization} + {\underbrace{γ {\sum }_{l > k} {W}_{kl} {‖ {Θ}_{k} - {Θ}_{l} ‖}_{{ℓ}_{q}}^{q}}}_{spatial regularization}\end{matrix} \qquad ( Elem - q )$$

Aspect 22. The method of Aspect 1, wherein the regularization stage is a trained machine learning model.

Aspect 23. The method of Aspect 1, further comprising generating a digital report of the one or more output inferred regulatory networks.

Aspect 24. The method of Aspect 1, further comprising analyzing the one or more output inferred regulatory networks against a set of treatments for upregulating or downregulating one or more of primary regulators in the output inferred regulatory networks; determining a subset of potential treatments based on the analysis; and generating a report identifying the subset or combinations of potential treatments.

Aspect 25. The method of Aspect 1, further comprising; applying the one or more output inferred regulatory networks to an enrichment process configured to assess output inferred regulatory network against one or more molecular functions, one or more biological processes, or one or more cellular components; and generating an enrichment report characterizing a subset of output inferred regulatory networks against the one or more molecular functions, the biological processes, and/or cellular components.

Aspect 26. The method of Aspect 25, wherein the enrichment report is an ontology enrichment report.

Aspect 27. The method of Aspect 25, wherein the sample is a tumor sample comprising cells from one or more of a glioblastoma, an anal cancer, a basal cell skin cancer, a squamous cancer, a benign cancer, a brain cancer, a glioblastoma, a breast cancer, a bladder cancer, a cervical cancer, a colon cancer, a colorectal cancer, an endometrial cancer, an esophageal cancer, a head and neck cancer, a liver cancer, a hepatobiliary cancer, a kidney cancer, a renal cancer, a gastric cancer, a gastrointestinal cancer, a lung cancer, a non-small cell lung cancer (NSCLC), a mesothelial cancer of the pleural cavity, a mesothelioma, an ovarian cancer, a pancreatic cancer, a prostate cancer, a rectal cancer, a lymphoma, a melanoma, a skin cancer, a meningioma, a sarcoma, and a thymus cancer.

Aspect 28. A non-transitory computer-readable storage medium storing executable instructions that, when executed by a processor, cause a computer to: obtain, at one or more processors, the structured-omic data for a sample, the structured-omic data comprising expression data and corresponding location data; perform, at the one or more processors, a clustering process on the structured-omic data to generate a plurality of candidate clusters, each candidate cluster corresponding to a different regulatory network of a subpopulation in the structured-omic data, each candidate cluster comprising a plurality of nodes and edges between nodes, each node corresponding to a regulator and each edge corresponding to an interaction between regulators connected by each edge; for each candidate cluster, generate a precision matrix, at the one or more processors, establishing a plurality of candidate precision matrices; provide, at the one or more processors, the plurality of candidate precision matrices to a regularization stage, and in the regularization stage comparing the candidate precision matrices using an optimization that infers one or more output precision matrices each output precision matrix corresponding to different subpopulations in the structured-omic data, wherein the regularization stage is tunable to one or more regularization processes defining the optimization and tunable to select a weighting factor imposing a similarity between candidate precise matrices that is applied during the optimization; and convert, at the one or more processors, the one or more output precision matrices to one or more output inferred regulatory networks.

Aspect 29. A computing system for inferring a regulatory network of interactions of regulators in structured-omic data, the computing system comprising: one or more processors; and one or more memories having stored thereon computer-executable instructions that, when executed, cause the computing system to: obtain, at one or more processors, the structured-omic data for a sample, the structured-omic data comprising expression data and corresponding location data; perform, at the one or more processors, a clustering process on the structured-omic data to generate a plurality of candidate clusters, each candidate cluster corresponding to a different regulatory network of a subpopulation in the structured-omic data, each candidate cluster comprising a plurality of nodes and edges between nodes, each node corresponding to a regulator and each edge corresponding to an interaction between regulators connected by each edge; for each candidate cluster, generate a precision matrix, at the one or more processors, establishing a plurality of candidate precision matrices; provide, at the one or more processors, the plurality of candidate precision matrices to a regularization stage, and in the regularization stage comparing the candidate precision matrices using an optimization that infers one or more output precision matrices each output precision matrix corresponding to different subpopulations in the structured-omic data, wherein the regularization stage is tunable to one or more regularization processes defining the optimization and tunable to select a weighting factor imposing a similarity between candidate precise matrices that is applied during the optimization; and convert, at the one or more processors, the one or more output precision matrices to one or more output inferred regulatory networks.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. Of course, the software or hardware descriptions herein may be implemented in hybrid deployments relying on both software/hardware, cloud-based deployments, etc. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. In some examples, the one or more processors may be implemented in hybrid-based or cloud-based processing environments.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as providing examples only and does not describe every possible embodiment, as describing every possible embodiment would be impractical, if not impossible. One could implement numerous alternate embodiments, using either current technology or technology developed after the filing date of this application.

What is claimed is:

1. A method of inferring a regulatory network of interactions of regulators in structured-omic data, the method comprising:
obtaining, at one or more processors, the structured-omic data for a sample, the structured-omic data comprising expression data and corresponding location data;
performing, at the one or more processors, a clustering process on the structured-omic data to generate a plurality of candidate clusters, each candidate cluster corresponding to a different regulatory network of a subpopulation in the structured-omic data, each candidate cluster comprising a plurality of nodes and edges between nodes, each node corresponding to a regulator and each edge corresponding to an interaction between regulators connected by each edge;
for each candidate cluster, generating a precision matrix, at the one or more processors, establishing a plurality of candidate precision matrices;
providing, at the one or more processors, the plurality of candidate precision matrices to a regularization stage, and in the regularization stage comparing the candidate precision matrices using an optimization that infers one or more output precision matrices each output precision matrix corresponding to different subpopulations in the structured-omic data, wherein the regularization stage is tunable to one or more regularization processes defining the optimization and tunable to select a weighting factor imposing a similarity between candidate precise matrices that is applied during the optimization; and
converting, at the one or more processors, the one or more output precision matrices to one or more output inferred regulatory networks.

2. The method of claim 1, wherein the regulator of each node of the candidate clusters is a biological regulator selected from the group consisting of DNA, RNA, a gene, a protein, proteome, a metabolite, or any combination thereof.

3. The method of claim 1, wherein the regulator of each node of the candidate clusters is a biological regulator of selected from the group consisting of genomic data, epigenomic data, chromatin state data, transcriptomic data, proteomic data, metabolomics data, spatial multiplexed imaging data or any combination thereof.

4. The method of claim 1, wherein structured-omic data comprises RNASeq data, spatially encoded RNASeq data, mRNA data, DNA data, miRNA, epigenomic data, proteomic data, spatial transcriptomic-epigenomic data, integrated transcriptomic-proteomic data, integrated multiplexed IF-transcriptomic data, or spatial Clustered Regularly Interspaced Short Palindromic Repeats (CRISPR)/CRISPR Associated (CAS) screens.

5. The method of claim 1, wherein each of the candidate clusters is a gene regulatory network and wherein each of the one or more output inferred regulatory networks is a gene regulatory network.

6. The method of claim 1, wherein each of the one or more output inferred regulatory networks corresponds to a spatially different subpopulation in the sample, a temporally different subpopulation in the sample, or a spatially-temporally different subpopulation in the sample.

7. The method of claim 1, wherein the sample comprises tissue taken from different organs within a subject.

8. The method of claim 1, wherein the sample comprises cancer tissue.

9. The method of claim 8, wherein the sample comprises primary cancer tissue and secondary cancer tissue.

10. The method of claim 1, wherein the one or more output inferred regulatory networks differ in a molecular function, a biological process, or cellular components.

11. The method of claim 1, wherein the interaction between regulators is an activation interaction or a suppression interaction between nodes defining the edge.

12. The method of claim 1, wherein the regularization stage comprises a plurality of regularization processes.

13. The method of claim 1, wherein the one or more regularization processes comprise a domain-informed regularization process, knowledge-informed regularization process, a platform-informed regularization process, structure-informed regularization process, a physics-informed regularization process.

14. The method of claim 1, wherein the regularization stage comprises a spatial regularization process or a temporal regularization process.

15. The method of claim 1, wherein the regularization stage comprises a smoothly-changing regularization process and a sparsely-changing regularization process.

16. The method of claim 15, wherein the smoothly-changing regularization process is an Q regularization process that is tunable by selecting A, y, and II values.

17. The method of claim 15, wherein the sparsely-changing regularization process is an I1 regularization process that is tunable by selecting A, y, and II values.

18. The method of claim 1, wherein the regularization stage comprises a backward mapping deviation as an Q regularization, an absolute regularization as an I1 regularization, and a tunable spatial regularization.

19. The method of claim 1, wherein the regularization stage performs a decomposability process prior to the optimization.

20. The method of claim 1, wherein the regularization stage is tunable through (i) a q tuning factor having values corresponding to the one or more regularization processes and (ii) a weighting factor for imposing a spatial similarity between candidate precision matrices.

21. The method of claim 1, wherein the regularization stage applies the optimization using the expression:
$$\begin{matrix}{\{ {\hat{Θ}}_{k} \}}_{k = 1}^{K} = arg {min}_{{\{ {\hat{Θ}}_{k} \}}_{k = 1}^{K}} {\underbrace{{\sum }_{k = 1}^{K} {‖ {Θ}_{k} - {\tilde{F}}^{*} ( {\hat{\sum }}_{k} ) ‖}_{{ℓ}_{2}}^{2}}}_{backward mapping deviation} \\ + {\underbrace{μ {\sum }_{k = 1}^{K} {‖ {Θ}_{k} ‖}_{{ℓ}_{1}}}}_{absolute regularization} + {\underbrace{γ {\sum }_{l > k} {W}_{kl} {‖ {Θ}_{k} - {Θ}_{l} ‖}_{{ℓ}_{q}}^{q}}}_{spatial regularization}\end{matrix} \qquad ( Elem - q )$$

22. The method of claim 1, wherein the regularization stage is a trained machine learning model.

23. The method claim 1, further comprising generating a digital report of the one or more output inferred regulatory networks.

24. The method claim 1, further comprising analyzing the one or more output inferred regulatory networks against a set of treatments for upregulating or downregulating one or more of primary regulators in the output inferred regulatory networks; determining a subset of potential treatments based on the analysis; and generating a report identifying the subset or combinations of potential treatments.

25. The method of claim 1, further comprising;
applying the one or more output inferred regulatory networks to an enrichment process configured to assess output inferred regulatory network against one or more molecular functions, one or more biological processes, or one or more cellular components; and
generating an enrichment report characterizing a subset of output inferred regulatory networks against the one or more molecular functions, the biological processes, and/or cellular components.

26. The method of claim 25, wherein the enrichment report is an ontology enrichment report.

27. The method of claim 1, wherein the sample is a tumor sample comprising cells from one or more of a glioblastoma, an anal cancer, a basal cell skin cancer, a squamous cancer, a benign cancer, a brain cancer, a glioblastoma, a breast cancer, a bladder cancer, a cervical cancer, a colon cancer, a colorectal cancer, an endometrial cancer, an esophageal cancer, a head and neck cancer, a liver cancer, a hepatobiliary cancer, a kidney cancer, a renal cancer, a gastric cancer, a gastrointestinal cancer, a lung cancer, a non-small cell lung cancer (NSCLC), a mesothelial cancer of the pleural cavity, a mesothelioma, an ovarian cancer, a pancreatic cancer, a prostate cancer, a rectal cancer, a lymphoma, a melanoma, a skin cancer, a meningioma, a sarcoma, and a thymus cancer.
